(19) United States
(12) Patent Application Publication
(10) Pub. No.: US 2023/0414537 A1
(43) Pub. Date: Dec. 28, 2023

(54) TOPICAL NAFTIFINE COMPOSITIONS

(71) Applicant: TARO PHARMACEUTICAL INDUSTRIES LTD., Haifa Bay (IL)

(72) Inventors: Helena SHIFRIN, Rehovot (IL); Alexandra SHRAIFEL, Nahariya (IL); Ron SCHLINGER, Tel Aviv (IL); Tzviel SHESKIN, Haifa (IL); Avi AVRAMOFF, Haifa (IL)

(21) Appl. No.: 18/338,860

(22) Filed: Jun. 21, 2023

Related U.S. Application Data
(60) Provisional application No. 63/366,728, filed on Jun. 21, 2022.

(51) Int. Cl. A61K 31/137; A61K 9/12; A61K 47/32; A61K 47/06; A61P 17/00; A61P 31/10; A61K 47/10; A61K 9/70; A61M 35/00

(52) U.S. Cl. CPC A61K 31/137; A61K 9/124; A61K 47/32; A61K 47/06; A61M 35/00; A61P 31/10; A61K 47/10; A61K 9/7015; A61P 17/00

(57) ABSTRACT

The present disclosure provides a topical sprayable composition comprising: (a) about 0.05% to about 5% by weight naftifine or pharmaceutical acceptable salt thereof, (b) a film forming agent comprising octylacrylamide acrylate copolymer and, (c) an alcoholic solvent, topical spray composition wherein the ratio of the alcoholic solvent to the propellant in the composition is about 1:1 to about 3:1 (w/w). The disclosure also provides a method of treating a dermatophyte skin infection or onychomycosis in a subject in need thereof, the method comprising topically administering to the subject the topical compositions as described herein.

### FIELD OF THE INVENTION

The present disclosure provides a topical sprayable composition comprising: (a) about 0.05% to about 5% by weight naftifine or pharmaceutical acceptable salt thereof, (b) a film forming agent comprising octylacrylamide acrylate copolymer and, (c) an alcoholic solvent, and (d) a propellant, wherein the ratio of the alcoholic solvent to the propellant in the composition is about 1:1 to about 3:1 (w/w). The disclosure also provides a drug-device combination for dispensing the compositions disclosed herein. Additionally, the disclosure also provides a method of treating a dermatophyte skin infection or onychomycosis in a subject in need thereof, the method comprising topically administering to the subject the topical spray composition as described herein.

### BACKGROUND

Dermatophyte skin infections are common in human populations. Tinea pedis, commonly known as “athlete's foot”, is a fungal, e.g., dermatophyte, infection of the soles and interdigital spaces of the feet. Symptoms include cracked, blistered or peeling areas of skin that causes itching, stinging, and burning. Tinea cruris is the dermatophyte infection that affects the genital, pubic, perineal, and perianal skin of the body. Common in athletes and people who sweat often in areas that trap moisture, Tinea cruris is also referred to as “jock itch”. The infection can cause redness, flakiness, peeling, or cracking of the skin of the groin area and a foul odor if the infection is severe or left untreated. While Tinea pedis and Tinea cruris are caused by a variety of fungi, the most common are Trichophyton rubrum, Trichophyton interdigitale, Tinea capitis and Epidermophyton floccosum, Tinea corporis, also known as “ringworm,” is the dermatophyte infection characterized by a red, itchy, and circular rash or lesion on the skin of any part of the body excluding the scalp, groin, palms, and soles. Dermatophyte infections can be recurring if not treated effectively and can lead to other serious medical conditions. Additionally, individuals who have at least one Dermatophyte infection on their bodies can develop other dermatophyte infections elsewhere on the body. Onychomycosis is a fungal infection of the nails that causes discoloration, thickening, and separation from the nail bed.

Currently, various creams and ointments are commercially available for the treatment of dermatophyte infections such as Tinea pedis and onychomycosis. Commercial formulations include the active ingredient Terbinafine (LAMISIL®) and Lamisil Once, Naftifine (NAFTIN®), and Luliconazole (LUZU®), as well as various azoles such as Clotrimazole. However, creams and ointments can be difficult to apply, are inconvenient, and can involve a messy application process.

### SUMMARY OF THE INVENTION

The present disclosure is directed to a sprayable topical composition comprising: (a) about 0.05% to about 5% by weight naftifine or pharmaceutical acceptable salt thereof, (b) a film forming agent comprising octylacrylamide acrylate copolymer; (c) an alcoholic solvent; and (d) a propellant, wherein the ratio of the alcoholic solvent to propellant in the composition is about 1:1 to about 3:1 (w/w).

In one embodiment, the present disclosure is directed to a topical composition comprising: (a) about 0.05% to about 5% by weight naftifine or pharmaceutical acceptable salt thereof, (b) a film forming agent; (c) an alcoholic solvent; and (d) a propellant, wherein the ratio of the alcoholic solvent to propellant in the composition is about 1:1 to about 3:1 (w/w).

In some embodiments, the naftifine or pharmaceutical acceptable salt thereof is naftifine hydrochloride. In some embodiments, the composition comprises about 1% to about 3% naftifine or a pharmaceutical acceptable salt thereof. In some embodiments, the composition comprises about 2% naftifine or a pharmaceutical acceptable salt thereof.

In some embodiments, the composition comprises about 2% to about 10% w/w octylacrylamide acrylate copolymer. In some embodiments, the composition comprises about 2% to about 4% w/w octylacrylamide acrylate copolymer. In some embodiments, the ratio of film forming agent to alcoholic solvent in the composition is about 1:15 to 1:30 (w/w). In some embodiments, the ratio of film forming agent to alcoholic solvent in the composition is about 1:18 to 1:22 (w/w).

In some embodiments, the composition comprises less than 4% hydrophilic polymer. In some embodiments, the composition comprises less than 0.5% hydrophilic polymer.

In some embodiments, the alcoholic solvent is a C₁-6 alcohol. In some embodiments, the C₁-6 alcohol is methanol, ethanol, propyl alcohol, n-propyl alcohol, iso-propyl alcohol, n-butyl alcohol, t-butyl alcohol, 2-butanol, iso-butanol, pentanol, hexanol, cyclohexanol, or combination thereof.

In some embodiments, the alcoholic solvent has a viscosity of about 1 cp to about 150 cp. In some embodiments, the composition comprises about 10% to about 70% by weight alcoholic solvent. In some embodiments, the composition comprises about 50% to about 70% by weight alcoholic solvent. In some embodiments, the composition comprises less than 70% by weight alcoholic solvent.

In some embodiments, the composition has a viscosity of about 0.1 cp to about 500 cp. In some embodiments, the composition has a viscosity of about 1 cp to about 150 cp.

In some embodiments, the propellant is a hydrocarbon propellant, a fluorocarbon propellant, a hydrofluorocarbon propellant, or combination thereof. In some embodiments, the propellant is propane, iso-butane, n-butane, isopentane, n-pentane, or combination thereof. In some embodiments, the propellant is a hydrofluorocarbon propellant. In some embodiments, the hydrofluorocarbon propellant is 1,1,1,2-Tetrafluoroethane (Norflurane 134A; HFA-134a) or 1,1-difluoroethane (HFA-152a). In some embodiments, the composition comprises about 10% to about 50% by weight propellant. In some embodiments, the composition comprises about 25% to about 45% by weight propellant.

In some embodiments, the ratio of the alcoholic solvent to the propellant is about 1.5:1 to about 2.5:1 (w/w). In some embodiments, the ratio of the alcoholic solvent to the propellant is about 1:1 to 2:1 (w/w). In some embodiments, the ratio of the alcoholic solvent to the propellant is about 2:1 to 3:1 (w/w). In some embodiments, the ratio of film forming agent to a propellant is about 1:7 to 1:15 (w/w). In some embodiments, the ratio of film forming agent to a propellant is about 1:8 to 1:12 (w/w).

In some embodiments, the composition further comprises a penetration enhancer, co-solvent, emulsifier, solubilizer, or combination thereof. In some embodiments, the penetration enhancer is diethylene glycol monoethyl ether, diethyl becacate, diisopropyl adipate, dimethyl isosorbide, dimethyl sulfoxide, polyethylene glycol, oleic acid, lactate esters, oleyl alcohol, alkyl glycoside, saccharide alkyl ester, dimethyl formamide, isopropyl myristate, Tween 80, sodium lauryl sulfate, a mixture of oleic acid and octyl dimethyl para-amino benzoic acid, or combination thereof. In some embodiments, the penetration enhancer is a combination of propylene glycol and diethylene glycol monoethyl ether. In some embodiments, the composition comprises about 0.1% to about 7% (w/w) penetration enhancer. In some embodiments, the composition comprises about 0.5% to about 2.5% (w/w) penetration enhancer.

In some embodiments, the disclosure provides a sprayable topical composition comprising: (a) about 0.05% to about 5% by weight naftifine or pharmaceutical acceptable salt thereof; (b) a film forming agent comprising octylacrylamide acrylate copolymer; (c) about 50% to about 75% by weight alcoholic solvent; and (d) about 20% to about 45% by weight propellant.

In some embodiments, the disclosure provides a sprayable topical composition comprising: (a) about 0.05% to about 5% by weight naftifine or pharmaceutical acceptable salt thereof, (b) about 1% to about 5% by weight of a film forming agent comprising octylacrylamide acrylate copolymer, (c) about 25% to about 45% by weight propellant, (d) about 50% to about 70% by weight alcoholic solvent, and (e) about 1% to about 3.5% by weight penetration enhancer.

In one embodiment, the disclosure provides a sprayable topical composition comprising: (a) about 0.05% to about 5% by weight naftifine or pharmaceutical acceptable salt thereof, (b) about 1% to about 5% by weight of a film forming agent, (c) about 25% to about 45% by weight propellant, (d) about 50% to about 70% by weight alcoholic solvent, and (e) about 1% to about 3.5% by weight penetration enhancer.

In some embodiments, the disclosure provides a delivery device comprising the topical composition as described herein. In some embodiments, the delivery device is a sealed and pressurized device. In some embodiments, the pressure in the sealed and pressurized device is about 28 psi to about 145 psi at 25° C. In some embodiments, the delivery device comprises a means for providing an aerosol spray of the topical composition. In some embodiments, the delivery device comprises a means for providing a non-aerosol spray of the topical composition.

In some embodiments, the disclosure provides a product of manufacture for the treatment of a dermatophyte skin infection or onychomycosis in a subject in need thereof, comprising (a) a sealed, pressurized container comprising (i) the topical composition as described herein, and (ii) means for providing an aerosol spray of the topical composition, and (b) instructions for use of the topical composition for treating the dermatophyte skin infection or onychomycosis.

In some embodiments, the disclosure provides a method of treating a dermatophyte skin infection or onychomycosis in a subject in need thereof, the method comprising topically administering to the subject the topical composition as described herein. In some embodiments, the dermatophyte skin condition is Tinea pedis or interdigital Tinea pedis or Tinea cruris. In some embodiments, the administering comprises applying the topical composition to the subject in an aerosol spray.

In some embodiments, the composition is administered not more than once in a three day period. In some embodiments, the composition is administered not more than once in a week period. In some embodiments, the composition is administered not more than once in a two week period. In some embodiments, the topical composition is administered not more than two times to the subject to alleviate the dermatophyte skin condition or onychomycosis. In some embodiments, the topical composition is administered once to the subject to alleviate the dermatophyte skin condition or onychomycosis. In some embodiments, the method comprises a first administration of the topical composition and a second administration of the topical composition. In some embodiments, the second composition is 5 days to 10 days after the first administration. In some embodiments, the second composition is about 7 days after the first administration.

In some embodiments, the effectiveness of treatment of the dermatophyte skin condition is determined by (a) comparing the clinical signs and symptoms associated with Tinea pedis in (i) a subject treated with the topical composition to; (ii) a subject treated with a placebo treatment; and/or (b) comparing mycological cure rate (i.e., potassium hydroxide stain and fungal culture results) in (i) a subject treated with the topical composition to; (ii) a subject treated with a placebo. In some embodiments, the systemic average levels of naftifine following a single application of the topical spray composition are about 100 pg/mL after 4 weeks.

In some embodiments, the disclosure provides a method of treating a dermatophyte skin infection or onychomycosis in a patient, the method comprising administering two doses of a topical composition comprising naftifine to the subject, wherein the topical composition is a monophasic solution and the naftifine is present in at least 80% saturation under conditions of use, wherein the second dose is administered about 1 week after first dose. In some embodiments, the dermatophyte skin condition is Tinea pedis or interdigital Tinea pedis or Tinea cruris.

In some embodiments, the disclosure provides a method of treating a dermatophyte skin infection or onychomycosis in a patient, the method comprising administering one dose of a topical composition comprising naftifine to the subject, wherein the topical composition is a monophasic solution and the naftifine is present in at least 80% saturation under conditions of use, In some embodiments, the dermatophyte skin condition is Tinea pedis or interdigital Tinea pedis or Tinea cruris.

In some embodiments, the disclosure provides a method for maintaining a therapeutic level of naftifine in the stratum corneum of a subject above Minimum Inhibitory Concentration (MIC). MIC is the lowest concentration of an antimicrobial agent needed to inhibit the visible in-vitro growth of a challenge microorganism.

In some embodiments, the disclosure provides a method for maintaining a therapeutic level above MIC of naftifine in the stratum corneum in a subject for 1, 2, 4, or 8 weeks, the method comprising administering two doses of a topical composition comprising naftifine to the subject, wherein the composition is a monophasic solution and the pharmaceutical is present in at least 80% saturation under conditions of use, wherein the second dose is administered about 1 week after first dose. In some embodiments, the amount of naftifine in the stratum corneum of the subject four weeks after the first dose and three weeks following the second dose is on average half the amount extracted at the second dose.

In some embodiments, the disclosure provides a method for maintaining a therapeutic level above MIC of naftifine in the stratum corneum in a subject for 1, 2, 4, or 8 weeks, the method comprising administering a single dose of a topical composition comprising naftifine to the subject, wherein the composition is a monophasic solution and the pharmaceutical is present in at least 80% saturation under conditions of use.

In some embodiments, the disclosure provides a method of treating a dermatophyte skin infection or onychomycosis in a subject in need thereof, the method comprising administering a sprayable topical composition comprising 2% naftifine or its pharmaceutically acceptable salt, wherein the topical composition is administered to the patient in one or two doses, wherein if two doses are used, the second dose is administered about 1 week after the first dose, wherein the administration of the topical composition to the subject results in at least 10%, 20%, 30%, 40% or 50% success in an efficacy endpoint. In some embodiments, the dermatophyte skin condition is Tinea pedis or interdigital Tinea pedis or Tinea cruris. In some embodiments, efficacy is measured using mycological cure (KOH and fungal culture), therapeutic effectiveness (defined as negative KOH stain, negative fungal culture and absent or mild erythema, scaling and pruritus) and clinical cure (defined as the absence of erythema, scaling and pruritus. In some embodiments, the primary efficacy endpoint is achieving Complete Clearance at Day 42 (6 weeks) defined as negative KOH, negative fungal culture, and the absence of the individual signs and symptoms (i.e., erythema, scaling and pruritus). In some embodiments, a percentage of subjects being administered the topical composition achieves a primary efficacy endpoint that is double a percentage of subjects being administer a placebo or not being treated.

In some embodiments, the disclosure provides a method of treating a dermatophyte skin infection or onychomycosis in a subject, the method comprising administering a topical composition comprising naftifine, wherein the composition is a monophasic solution and the pharmaceutical is present in at least 80% saturation under conditions of use, wherein the administration of the topical composition to the subject results in a complete clearance of symptoms with improvement in associated parameters selected from: (a) a decrease in severity of erythema from baseline with a score of 0 on day 42; (b) a decrease in severity of scaling from baseline with a score of 0 on day 42; (c) a decrease in severity of pruritus from baseline with a score of 0 on day 42; or combinations thereof. In some embodiments, the dermatophyte skin condition is Tinea pedis or interdigital Tinea pedis or Tinea cruris.

In some embodiments, the disclosure provides a method of treating a dermatophyte skin infection in a subject, the method comprising administering a topical sprayable composition comprising naftifine, wherein the composition is a monophasic solution and the pharmaceutical is present in at least 80% saturation under conditions of use, wherein the administration of the topical composition to the subject results in a therapeutic effectiveness rate of at least 20%, wherein the Therapeutic Effectiveness is a negative KOH stain, negative fungal culture and absent or mild erythema, scaling and pruritus six weeks after the treatment.

In some embodiments, the method comprises administering a single dose of the topical sprayable composition. In some embodiments, the method comprises administering two doses of the topical sprayable composition. In some embodiments, the administration of the topical composition to the subject results in a therapeutic effectiveness rate of at least 40%. In some embodiments, the administration of the topical composition to the subject results in a therapeutic effectiveness rate of at least 70%.

In some embodiments, the disclosure provides a drug-device combination for dispensing a composition, the drug-device combination comprising a container comprising a composition comprising naftifine or pharmaceutical acceptable salt thereof, a film forming agent comprising octylacrylamide acrylate copolymer, an alcoholic solvent, and a propellant, wherein the drug-device combination is suitable for providing a spray comprising the composition, wherein the spray has a D₉₀ droplet size greater than 1 μm.

In some embodiments, the composition of the drug-device combination has a D₉₀ droplet size of about 140 μm to about 170 μm when sprayed from the device. In some embodiments, the composition of the drug-device combination has a D₉₀ droplet size of about 150 μm to about 165 μm when sprayed from the device. In some embodiments, the composition of the drug-device combination has an average D₉₀ droplet size of about 155 μm to 163 μm when sprayed from the device.

In some embodiments, the composition of the drug-device combination comprises naftifine or pharmaceutical acceptable salt thereof is naftifine hydrochloride. In some embodiments, the composition of the drug-device combination comprises about 1% to about 3% naftifine or a pharmaceutical acceptable salt thereof. In some embodiments, the composition of the drug-device combination comprises about 2% naftifine or a pharmaceutical acceptable salt thereof.

In some embodiments, the composition of the drug-device combination comprises about 2% to about 10% w/w octylacrylamide acrylate copolymer. In some embodiments, the composition of the drug-device combination comprises about 2% to about 4% w/w octylacrylamide acrylate copolymer. In some embodiments, the composition of the drug-device combination comprises a composition, wherein the ratio of film forming agent to alcoholic solvent in the composition is about 1:15 to 1:30 (w/w). In some embodiments, the composition of the drug-device combination comprises a composition, wherein the ratio of film forming agent to alcoholic solvent in the composition is about 1:18 to 1:22 (w/w).

In some embodiments, the composition of the drug-device combination comprises less than 4% hydrophilic polymer. In some embodiments, the composition of the drug-device combination comprises a composition comprising less than 0.5% hydrophilic polymer.

In some embodiments, the alcoholic solvent of the composition of the drug-device combination is a C₁-C₆ alcohol. In some embodiments, the C₁-C₆ alcohol of the composition of the drug-device combination is methanol, ethanol, propyl alcohol, n-propyl alcohol, iso-propyl alcohol, n-butyl alcohol, t-butyl alcohol, 2-butanol, isobutanol, pentanol, hexanol, cyclohexanol, or combination thereof. In some embodiments, the C₁-C₆ alcohol of the composition of the drug-device combination is ethanol.

In some embodiments, the alcoholic solvent of the composition of the drug-device combination has a viscosity of about 1 cp to about 150 cp. In some embodiments, the composition of the drug-device combination comprises about 10% to about 70% by weight alcoholic solvent. In some embodiments, the composition of the drug-device combination comprises about 50% to about 70% by weight alcoholic solvent.

In some embodiments, the drug-device combination comprises a composition, wherein the composition has a viscosity of about 0.1 cp to about 500 cp. In some embodiments, the drug-device combination comprises a composition, wherein the composition has a viscosity of about 1 cp to about 150 cp.

In some embodiments, the propellant of the composition of the drug-device combination is a hydrocarbon propellant, a fluorocarbon propellant, a hydrofluorocarbon propellant, or combination thereof. In some embodiments, the propellant of the composition of the drug-device combination is propane, iso-butane, n-butane, isopentane, n-pentane, or combination thereof. In some embodiments, the propellant of the composition of the drug-device combination is a hydrofluorocarbon propellant. In some embodiments, the hydrofluorocarbon propellant of the composition of the drug-device combination is 1,1,1,2-Tetrafluoroethane (Norflurane 134A; HFA-134a) or 1,1-difluoroethane (HFA-152a).

In some embodiments, the composition of the drug-device combination comprises about 10% to about 50% by weight propellant. In some embodiments, the composition of the drug-device combination comprises about 25% to about 45% by weight propellant.

In some embodiments, the drug-device combination comprises a composition, wherein the ratio of the alcoholic solvent to the propellant is about 1.5:1 to about 2.5:1 (w/w). In some embodiments, the drug-device combination comprises a composition, wherein the ratio of the alcoholic solvent to the propellant is about 1:1 to 2:1 (w/w). In some embodiments, the drug-device combination comprises a composition, wherein the ratio of the alcoholic solvent to the propellant is about 2:1 to 3:1 (w/w). In some embodiments, the drug-device combination comprises a composition, wherein the ratio of film forming agent to a propellant is about 1:7 to 1:15 (w/w). In some embodiments, the drug-device combination comprises a composition, wherein the ratio of film forming agent to a propellant is about 1:8 to 1:12 (w/w). In some embodiments, the composition of the drug-device combination further comprises a penetration enhancer, co-solvent, emulsifier, solubilizer, or combination thereof.

In some embodiments, the penetration enhancer of the composition of the drug-device combination is diethylene glycol monoethyl ether, diethyl becacate, diisopropyl adipate, dimethyl isosorbide, dimethyl sulfoxide, polyethylene glycol, oleic acid, lactate esters, oleyl alcohol, alkyl glycoside, saccharide alkyl ester, dimethyl formamide, isopropyl myristate, Tween 80, sodium lauryl sulfate, a mixture of oleic acid and octyl dimethyl paraamino benzoic acid, or combination thereof.

In some embodiments, the pressure inside the drug-device combination is about 28 psi to about 145 psi at 25° C.

In some embodiments, the disclosure provides a method of treating a dermatophyte skin infection or onychomycosis in a subject in need thereof, the method comprising applying an aerosol spray to the skin using the drug-device combination disclosed herein. In some embodiments, the dermatophyte skin condition is Tinea pedis or interdigital Tinea pedis or Tinea cruris. In some embodiments, the composition is administered not more than once in a three day period. In some embodiments, the composition is administered not more than once in a week period. In some embodiments, the composition is administered not more than once in a two week period. In some embodiments, the composition is administered not more than two times to the subject to alleviate the dermatophyte skin condition or onychomycosis. In some embodiments, the topical composition is administered once. In some embodiments, the composition is administered in one application.

In some embodiments, the composition dries less than three minutes after application of the composition to the skin. In some embodiments, the composition dries less than two minutes after application of the composition to the skin. In some embodiments, the composition dries less than one minute after application of the composition to the skin.

In some embodiments, the method comprises a first administration of the topical composition and a second administration of the composition. In some embodiments, the second administration is 5 days to 10 days after the first administration. In some embodiments, the second administration is about 7 days after the first administration.

### DETAILED DESCRIPTION OF THE INVENTION

The present disclosure relates to compositions and methods suitable for the treatment of a dermatophyte skin infection, e.g., Tinea pedis, interdigital Tinea pedis Tinea cruris and Tinea corporis or onychomycosis. In some embodiments, the disclosure provides a topical composition comprising naftifine, e.g., a topical aerosol spray. Naftifine cream has been used previously in the treatment of dermatophyte infections such as Tinea pedis, Tinea cruris and Tinea corporis. However, the present disclosure provides for a new composition which is fast-acting, timely and effective treatment with limited/reduced side effects against dermatophyte infections and onychomycosis. The topical compositions of the present invention are also non-greasy, and dry quickly. The present disclosure also provides compositions effective for the treatment of dermatophyte infections such as Tinea pedis, interdigital Tinea pedis Tinea cruris and Tinea corporis with little blood absorption, and thus have a low systemic effect. The topical compositions provided herein have decreased erythema, scaling and puritus of the skin. Additionally, the present disclosure provides compositions for treating dermatophyte infections such as Tinea pedis, interdigital Tinea pedis Tinea cruris, Tinea corporis, and Tinea capitis which are effective when applied in a reduced number of doses, e.g., when applied once or twice to a subject in need thereof. In some embodiments, the sprayable compositions described herein can be sprayed directly on the skin surface, and do not need to be spread over the skin by touching with fingers or with a separate applicator, avoiding touching the dermatophyte affected area with non-infected body parts. In some embodiments, direct application to the skin via a spray provides for increased convenience, and lower transmission of the naftifine to the hands/fingers or other body parts for which it is not needed. In some embodiments, the sprayable compositions described herein can be applied quickly. In some embodiments, the sprayable compositions described herein can be efficiently applied to areas which are less accessible, such as the interdigital spaces. In some embodiments, the sprayable compositions described herein can be applied quickly over a large area of the skin.

Although terbinafine is more soluble in alcohol than naftifine, surprisingly the topical naftifine composition of the present invention is fully solubilized in the formulation and achieves skin permeability and skin retention which allows clinical anti-fungal effect following single application as obtained with the terbinafine Once product.

Unless otherwise defined herein, scientific and technical terms used in the present disclosure shall have the meanings that are commonly understood by one of ordinary skill in the art. Further, unless otherwise required by context, singular terms shall include pluralities and plural terms shall include the singular. As used herein, “a” or “an” may mean one or more. As used herein, when used in conjunction with the word “comprising,” the words “a” or “an” may mean one or more than one. As used herein, “another” or “a further” may mean at least a second or more.

Throughout this application, the term “about” is used to indicate that a value includes the inherent variation of error for the method/device being employed to determine the value, or the variation that exists among the study subjects. Typically, the term “about” is meant to encompass approximately or less than 1%, 2%, 3%, 4%, 5%, 6%, 7%, 8%, 9%, 10%, 11%, 12%, 13%, 14%, 15%, 16%, 17%, 18%, 19% or 20% variability, depending on the situation.

The use of the term “or” in the claims is used to mean “and/or”, unless explicitly indicated to refer only to alternatives or the alternatives are mutually exclusive, although the disclosure supports a definition that refers to only alternatives and “and/or.”

As used herein, the terms “comprising” (and any variant or form of comprising, such as “comprise” and “comprises”), “having” (and any variant or form of having, such as “have” and “has”), “including” (and any variant or form of including, such as “includes” and “include”) or “containing” (and any variant or form of containing, such as “contains” and “contain”) are inclusive or open-ended and do not exclude additional, unrecited, elements or method steps. It is contemplated that any embodiment discussed in this specification can be implemented with respect to any protein, compositions, polynucleotides, vectors, cells, methods, and/or kits of the present disclosure. Furthermore, compositions, polynucleotides, vectors, cells, and/or kits of the present disclosure can be used to achieve methods and proteins of the present disclosure.

The use of the term “for example” and its corresponding abbreviation “e.g.,” (whether italicized or not) means that the specific terms recited are representative examples and embodiments of the disclosure that are not intended to be limited to the specific examples referenced or cited unless explicitly stated otherwise.

As used herein, “between” is a range inclusive of the ends of the range. For example, a number between x and y explicitly includes the numbers x and y, and any numbers that fall within x and y.

The present disclosure provides a sprayable topical compositions comprising naftifine, e.g., topical spray compositions. In some embodiments, the disclosure provides a topical composition comprising: (a) about 0.05% to about 5% by weight naftifine or pharmaceutical acceptable salt thereof, (b) a film forming agent comprising octylacrylamide acrylate copolymer and, (c) an alcoholic solvent, wherein the ratio of the alcoholic solvent to the propellant in the composition is about 1:1 to about 3:1 (w/w).

In one embodiment, the film forming agent may be one or more of polyvinyl pyrrolidone, polyvinyl alcohol, acrylic polymers, acrylic copolymers, methacrylate polymers, octylacrylamide acrylate copolymers, methacrylate copolymers, poly (vinyl acetate), cellulose based polymers, cellulose based co-polymers, pyroxylin (cellulose nitrate), Pullulan (polysaccharide polymer), alginates.

Naftifine can be represented by Formula I.

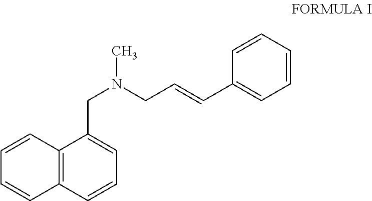 [SMILES: CN(C/C=C/c1ccccc1)Cc1cccc2ccccc12]

As used herein, the term naftifine includes the compound of Formula I, as well as salts, solvates, and stereoisomers thereof. For example, in some embodiments, naftifine includes naftifine hydrochloride, as well as other pharmaceutically acceptable salts.

The concentration of naftifine can vary in the composition. In some embodiments, the concentration of naftifine is less than 5% by weight of the composition. In some embodiments, the concentration of naftifine is less than 4.5%, less than 4.0%, less than 3.5%, or less than 3.0% by weight of the composition. In some embodiments, the composition comprises about 0.05% to about 5% by weight naftifine. In some embodiments, the composition comprises about 0.05% to about 4%, about 0.1% to about 3.5%, about 0.1% to about 3.0%, about 0.2% to about 3.0%, about 0.5% to about 2.5%, about 1.0% to about 3.0%, about 1.0% to about 2.5%, about 1.5% to about 2.5%, about 1.8% to about 2.2% or about 2.0% by weight naftifine.

The present disclosure provides a topical composition for administration. In some embodiments, the present disclosure provides a topical aerosol spray composition for administration. In some embodiments, the term topical aerosol spray composition is a pressurized composition, e.g., a composition in a closed container under pressure, such that an aerosol can be formed upon release form the container. In some embodiments, the topical composition can include a composition with or without being pressurized. For example, the term “topical composition” can include (i) a liquid composition inside a pressurized container, which becomes an aerosol spray when discharged from the container, and (2) the aerosol spray discharged from the container. Additionally, the term “topical composition” in some embodiments can include the liquid composition on a surface, e.g., a skin surface, which forms when the aerosol spray comes in contact with the surface. In some embodiments, administration by aerosol spray provides a more consistent administration of the composition on the subject being treated. In some embodiments, administration by aerosol spray provides a more thorough administration of the composition to the subject, e.g., more area is covered. In some embodiments, administration by aerosol spray provides a more convenient method of administration. In some embodiments, administration by aerosol spray reduces the need for other applicators, e.g., wipes, gauzes, clothes, etc. In some embodiments, administration by aerosol spray reduces exposure of the hands or other unintended body parts to the presence of naftifine that may occur by administration by creams, gels, or liquids. In some embodiments, administration by aerosol spray provides a more convenient mode of administration, as it allows the composition to dry quickly after application.

In some embodiments, treatment of the dermatophyte skin infection or onychomycosis comprises applying the topical composition to the subject in an aerosol spray. In some embodiments, the topical composition is a monophasic solution. In some embodiments, the topical composition is a saturated, monophasic solution as outlined in U.S. Pat. No. 8,349,297, incorporated by reference herein in its entirety. The term “monophasic” as used herein indicates that the composition does not contain undissolved component (such as naftifine or any other excipient), and that there is only the one liquid phase, and not a colloid or micro-colloid composition. Thus, in some embodiments, the topical composition only has one phase, and the phase is liquid.

In some embodiments, the term “saturated” can include substantially saturated, wherein at least 80% of that amount, at least 90%, or at least 95% of that amount of naftifine needed to achieve saturation is present. In some embodiments, at the temperature of use, the composition is as close to saturated as possible, while remaining monophasic. The term “saturated” can also include supersaturated solutions under certain conditions.

In some embodiments, the amount of naftifine is as close to full saturation as possible. In some embodiments, the addition of antinucleating agents may be advantageous, as may a slight drop in saturation, down as far as 80%, which can be considered to be a saturating amount for the purposes of the present invention.

The present disclosure provides for naftifine composition comprising a film forming agent. The disclosure demonstrates that in topical compositions, e.g., topical spray compositions, a film forming agent comprising octylacrylamide acrylate copolymer provide for a longer-lasting effect to the treated area, while minimizing systemic exposure of naftifine. In some embodiments, the long lasting effect of the present compositions allow for a reduced number of total administrations of the naftifine composition. In some embodiments, the long lasting effect of the present compositions allow for a reduced frequency of administration of the naftifine composition.

In some embodiments, the composition comprises a film forming agent comprising octylacrylamide acrylate copolymer. The term octylacrylamide acrylate copolymer refers to an octylacrylamide polymer with one or more monomers of acrylic acid, methacrylic acid or one of their simple esters. Various octylacrylamide acrylate copolymers are known in the art, and can include Octylacrylamide/acrylates/butylaminoethyl methacrylate copolymer. In some embodiments, the octylacrylamide acrylate copolymer is DERMACRYL® Polymer (Nouryon), a hydrophobic, high molecular weight carboxylated acrylic copolymer. DERMACRYL® is also known as Acrylates-t-octylpropenamide. In some embodiments the octylacrylamide acrylate copolymer is Dermacryl LT (carboxylated acrylic copolymer of acrylates and octylacrylamide). In some embodiments the octylacrylamide acrylate copolymer is Dermacryl E (styrene \acrylate copolymer). In some embodiments, the topical composition can have more than one film-forming agent. In some embodiments, the octylacrylamide acrylate copolymer comprises greater than 50%, greater than 60%, greater than 70%, greater than 80%, greater than 90% or greater than 95% of the film forming agents in the topical composition. In some embodiments, the topical composition comprises octylacrylamide acrylate copolymer as the sole film forming agent.

Various concentrations of the film forming agent are present in the topical composition. In some embodiments, the topical composition comprises less than 10%, less than 8%, less than 6%, less than 5%, or less than 4% by weight of the film forming agent. In some embodiments, the topical composition comprises greater than 0.5%, greater than 1.0%, greater than 1.5%, greater than 2.0%, or greater than 2.5% by weight of the film forming agent. In some embodiments, the topical composition comprises about 1% to about 10% by weight octylacrylamide acrylate copolymer. In some embodiments, the topical composition comprises about 1.5% to about 8.0%, about 1.5% to about 6.0%, about 2.0% to about 4.0%, or about 2.5% to about 3.5% by weight octylacrylamide acrylate copolymer.

The disclosure provides various ratios of film forming agent to alcoholic solvent in the topical composition. In some embodiments, these ratios provide for stable compositions suitable for solubilization of the naftifine while still being suitable for topical administration, e.g., by a spray, e.g., an aerosol spray or a mist spray. In some embodiments, the ratio of film forming agent to alcoholic solvent in the composition is about 1:10 to about 1:40 (w/w). In some embodiments, the ratio of film forming agent to alcoholic solvent in the composition is about 1:15 to 1:30, about 1:16 to about 1:25 or about 1:18 to 1:22 (w/w). In some embodiments, the ratio of film forming agent to alcoholic solvent in the composition is about 1:20.

The applicants of the present disclosure found that hydrophilic polymers previously used for dermal compositions were in some embodiments, not required in the compositions described herein. In some embodiments, the disclosure provides topical compositions with low concentrations of hydrophilic polymers, e.g., less than 5% hydrophilic polymers. In some embodiments, the disclosure provides topical compositions with no hydrophilic polymers. In some embodiments, the composition comprises less than 4%, less than 3%, less than 2%, less than 1%, or less than 0.5% by weight hydrophilic polymer. In some embodiments, the hydrophilic polymers reduced or excluded from the topical composition is polyvinylpyrrolidone, vinylpyrrolidone-vinylacetate copolymer, hydroxyalkyl cellulose and/or alkyl cellulose. In some embodiments, the topical compositions with reduced hydrophilic polymers (or no hydrophilic polymers) provide for increased physical stability of the topical composition. In some embodiments, the topical compositions with reduced hydrophilic polymers (or no hydrophilic polymers) provide for increased chemical stability of the topical composition. In some embodiments, the topical compositions with reduced hydrophilic polymers (or no hydrophilic polymers) provide for a viscosity suitable for a spray, e.g., an aerosol spray. In some embodiments, the topical compositions with reduced hydrophilic polymers (or no hydrophilic polymers) provide a spray which is more evenly dispersed, and does not clog the dispensing apparatus (e.g., an aerosol apparatus). In some embodiments, the topical compositions with reduced hydrophilic polymers (or no hydrophilic polymers) provide for a quicker drying time relative to compositions comprising hydrophilic polymers. In some embodiments, the topical compositions with reduced hydrophilic polymers (or no hydrophilic polymers) results in less residue on the skin of the subject being treated. In some embodiments, the topical compositions with reduced hydrophilic polymers (or no hydrophilic polymers) provide a composition that adheres to the skin for a period of time longer than compositions comprising a hydrophilic polymer. In some embodiments, the topical compositions with reduced hydrophilic polymers (or no hydrophilic polymers) provide for a more economical composition, such that the topical composition is easier to manufacture, quicker to manufacture, cheaper to manufacture, and/or requires less equipment to manufacture, thereby streamlining manufacturing relative to compositions which require hydrophilic polymers.

Various solvents can be used in the present invention. In some embodiments, the solvent is suitable for solubilizing naftifine, e.g., an organic solvent, which is pharmaceutically acceptable. In some embodiments, the solvent is an alcoholic solvent. In some embodiments, the alcoholic solvent is a C₁₋₆ alcohol. In some embodiments, the C₁₋₆ alcohol is methanol, ethanol, propyl alcohol, n-propyl alcohol, iso-propyl alcohol, n-butyl alcohol, t-butyl alcohol, 2-butanol, iso-butanol, pentanol, hexanol, cyclohexanol, or combination thereof. In some embodiments, the alcoholic solvent has a viscosity suitable for administering via an aerosol spray or a mist. In some embodiments, the alcoholic solvent has a viscosity of about 1 cp to about 150 cp.

In some embodiments, the composition comprises about 10% to about 80% by weight alcoholic solvent. In some embodiments, the composition comprises about 20% to about 80%, about 30% to about 80%, about 40% to about 80% or about 50% to about 80% by weight alcoholic solvent. In some embodiments, the composition comprises about 52% to about 68%, about 54% to about 66%, about 56% to about 64% or about 58% to about 62% by weight alcoholic solvent. In some embodiments, the composition comprises about 60% by weight alcoholic solvent. In some embodiments, the composition comprises less than 80%, less than 70%, less than 65%, less than 62% or less than 61% by weight of alcoholic solvent. In some embodiments, the composition comprises about 70% to about 85% alcoholic solvent, about 72% to about 82% alcoholic solvent, or about 74% to about 80% alcoholic solvent by weight. In some embodiments, the composition comprises about 75%, about 76%, about 77%, about 78%, about 79%, about 80%, about 81%, about 82% or about 83% alcoholic solvent by weight. In some embodiments, the reduced alcoholic solvent concentration can result in reduced adverse effect, e.g., inflammation or irritation.

In some embodiments, the composition comprises about 10% to about 70% by weight C₁₋₆ alcohol solvent. In some embodiments, the composition comprises about 20% to about 70%, about 30% to about 70%, about 40% to about 70% or about 50% to about 70% by weight C₁₋₆ alcohol solvent. In some embodiments, the composition comprises about 52% to about 68%, about 54% to about 66%, about 56% to about 64% or about 58% to about 62% by weight C₁₋₆ alcohol solvent. In some embodiments, the composition comprises about 70% to about 85% C₁₋₆ alcohol solvent, about 72% to about 82% C₁₋₆ alcohol solvent, or about 74% to about 80% C₁₋₆ alcohol solvent by weight. In some embodiments, the composition comprises about 75%, about 76%, about 77%, about 78%, about 79%, about 80%, about 81%, about 82% or about 83% C₁₋₆ alcohol solvent by weight. In some embodiments, the composition comprises about 60% by weight C₁₋₆ alcohol solvent.

In some embodiments, the disclosure provides a sprayable topical composition comprising: (a) about 0.05% to about 5% by weight naftifine or pharmaceutical acceptable salt thereof; (b) a film forming agent comprising octylacrylamide acrylate copolymer; (c) about 50% to about 80% by weight alcoholic solvent; and (d) about 15% to about 45% by weight propellant. In some embodiments, the disclosure provides a sprayable topical composition comprising: (a) about 0.05% to about 5% by weight naftifine or pharmaceutical acceptable salt thereof, (b) about 1% to about 5% by weight of a film forming agent comprising octylacrylamide acrylate copolymer, (c) about 15% to about 45% by weight propellant, (d) about 50% to about 80% by weight alcoholic solvent, and (e) about 1% to about 3.5% by weight penetration enhancer. In some embodiments, the disclosure provides a sprayable topical composition comprising: (a) about 0.05% to about 5% by weight naftifine or pharmaceutical acceptable salt thereof, (b) a film forming agent comprising octylacrylamide acrylate copolymer; (c) an alcoholic solvent; and (d) a propellant, wherein the time to film formation is less than about 30 seconds.

In some embodiments, the ratio alcoholic solvent to propellant is about 1:1 to about 3:1, about 1.1:1 to about 2.9:1, about 1.2:1 to about 2.8:1, about 1.3:1 to about 2.7:1, about 1.4:1 to about 2.6:1, about 1.5:1 to about 2.5:1, about 1.6:1 to about 2.4:1, about 1.7:1 to about 2.3:1, or about 1.8:1 to about 2.2:1.

In some embodiments, the topical composition has a viscosity suitable for administering via an aerosol spray and/or a mist. For example, in some embodiments, the topical composition has a viscosity of less than 1000 cp, less than 700 cp, less than 500 cp or less than 300 cp. In some embodiments, the topical formation has a viscosity of about 1 cp to about 500 cp, about 5 cp to about 300 cp or about 50 cp to about 250 cp. In some embodiments, the topical composition has a viscosity of about 1 cp to about 150 cp. As used throughout the present disclosure, viscosity is measured at standard conditions, e.g., 1 atm pressure and 25° C. temperature.

In some embodiments, the topical composition produces an aerosol when discharged from a pressurized container using a standard single fluid aerosol nozzle, e.g., a plain-orifice nozzle or a shaped-orifice nozzle, e.g., a “V” notch nozzle. In some embodiments, the composition is discharged at a pressure drop of about 20 bars to about 30 bars, e.g., about 25 bars (2,500 kPa; 360 psi) at room temperature. In some embodiments, the aerosol has a droplet size of about 10 μm to about 100 μm, In some embodiments, at least 99% of droplets are 100 μm and above in diameter. In some embodiments, at least 99% of droplets are 100 μm and below in diameter.

In some embodiments, the topical composition further comprises a penetration enhancer, co-solvent, emulsifier, propellant, solubilizer, or combination thereof. In some embodiments, the topical composition further comprises a propellant. In some embodiments, the propellant is a hydrocarbon, a fluorocarbon, a hydrofluorocarbon propellant, or combination thereof. In some embodiments, the propellant is propane, iso-butane, n-butane, isopentane, n-pentane, or combination thereof. In some embodiments, the propellant is a hydrofluorocarbon propellant. In some embodiments, the hydrofluorocarbon propellant is 1,1,1,2-Tetrafluoroethane (NORFLURANE® 134A, HFA-134a) or 1,1-difluoroethane (HFA-152a, ZEPHEX® 152a).

In some embodiments, the propellant provides for a high saturation level of naftifine. In some embodiments, the propellant is a highly volatile liquid with a low boiling point, such as a CFC or HFA, such that it can force the composition from a dispenser. In some embodiments, the propellant provides that evaporation is almost instantaneous and the boiling during transfer from the dispenser to the site of administration has the effect of causing the evaporation of a substantial amount of the solvent. Thus, in some embodiments, the solvent is preferably a volatile solvent, and is preferably more volatile than water and will often be organic. In some embodiments, the decompression of the propellant during administration causes the disruption and loss of solvent by evaporation. This loss can be up to 50% and even higher.

In some embodiments, the topical composition comprises various concentrations of propellant. In some embodiments, the concentration of propellant is suitable for providing an aerosol naftifine spray. In some embodiments, the concentration of propellant is suitable for providing a naftifine mist spray. In some embodiments, the composition comprises about 10% to about 50% by weight propellant. In some embodiments, the composition comprises about 25% to about 45% by weight propellant.

The disclosure provides that various ratios of film forming agent to propellant can be used in the topical composition. In some embodiments, the ratio of film forming agent to a propellant is about 1:7 to 1:15, about 1:7 to about 1:14, or about 1:8 to about 1:13 (w/w). In some embodiments, the ratio of film forming agent to propellant is about 1:8 to 1:12 (w/w).

The disclosure provides various ratios of the alcoholic solvent to the propellant used in the topical compositions described herein. In some embodiments, the ratio of the alcoholic solvent to the propellant is about 1:1 to about 3:1, about 1.5:1 to about 1:1, or about 2:1 to about 1:1 (w/w). In some embodiments, the ratio of the alcoholic solvent to the propellant is about 1:2 to about 1:3. In some embodiments, the ratio of alcoholic solvent to propellant is about 1.1:1, 1.2:1, 1.3:1, 1.4:1, 1.5:1, 1.6:1, 1.7:1, 1.8:1, 1.9:1, 2.0:1, 2.1:1, 2.2:1, 2.3:1, 2.4:1, 2.5:1, 2.6:1, 2.7:1, 2.8:1, or 2.9:1, 3:1 or any range containing any two of these ratios. In some embodiments, the disclosed ratio of alcoholic solvent to propellant provides a composition that is physically stable, e.g., no particles are formed, the composition is homogeneous, the naftifine is soluble, etc. In some embodiments, the disclosed ratio of alcoholic solvent to propellant provides a composition that is chemically stable, e.g., the naftifine does not degrade for an extended period of time, e.g., 3 months, 6 months, 9 months, 1 year, 18 months, and/or 2 years in RT conditions, or accelerated conditions.

In some embodiments, the topical composition can comprise a penetration enhancer. The term “penetration enhancer” refers to any compound or composition that interacts with the constituents of the skin's outermost and rate limiting layer stratum corneum (SC), and increase its permeability. Penetration enhancers can include substances which promote the absorption of naftifine into the skin temporarily by transiently enhancing the skin permeability. Penetration enhancers can be employed to transfer the delivery of naftifine, while minimizing the systemic delivery of naftifine in the blood. As used herein, the penetration enhancer can include diethylene glycol monoethyl ether, diethyl becacate, diisopropyl adipate, dimethyl isosorbide, dimethyl sulfoxide, polyethylene glycol, oleic acid, lactate esters, oleyl alcohol, alkyl glycoside, saccharide alkyl ester, dimethyl formamide, isopropyl myristate, Tween 80, sodium lauryl sulfate, a mixture of oleic acid and octyl dimethyl para-amino benzoic acid, or combination thereof. In some embodiments, one, two, three or greater than three penetration enhancers are in the topical composition. In some embodiments, two penetration enhancers are in the composition. In some embodiments, the penetration enhancer is a combination of propylene glycol and diethylene glycol monoethyl ether (TRANSCUTOL®).

Various concentration of penetration enhancer can be present in the topical composition. The penetration enhancer can be adjusted to facilitate the penetration of naftifine, while minimizing the systemic absorption. In some embodiments, the topical composition comprises about 0.1% to about 7%, about 0.2% to about 5%, about 0.5% to about 3%, or about 0.75% to about 3% by weight penetration enhancer. In some embodiments, the composition comprises about 0.5% to about 2.5% by weight penetration enhancer.

In some embodiments, the topical composition comprises two penetration enhancers, wherein each penetration enhancer is about 0.1% to about 4%, about 0.2% to about 3%, about 0.5% to about 2%, or about 0.5% to about 1.5% by weight of the topical composition. In some embodiments, the topical composition comprises one or both of propylene glycol and diethylene glycol monoethyl ether, wherein propylene glycol is about 0.1% to about 4%, about 0.2% to about 3%, about 0.5% to about 2%, or about 0.5% to about 1.5% by weight of the topical composition, and/or wherein diethylene glycol monoethyl ether is about 0.1% to about 4%, about 0.2% to about 3%, about 0.5% to about 2%, or about 0.5% to about 1.5% by weight of the topical composition. In some embodiments, the topical composition comprises about 1% to about 3.5% by weight penetration enhancer. In some embodiments, the topical composition comprises a combination of propylene glycol and diethylene glycol monoethyl ether, wherein propylene glycol is about 0.5% to about 2% by weight, and the diethylene glycol monoethyl ether is about 0.5% to about 2% by weight of the topical composition. In some embodiments, the topical composition comprises a combination of propylene glycol and diethylene glycol monoethyl ether, wherein propylene glycol is about 0.5% to about 1% by weight, and the diethylene glycol monoethyl ether is about 0.5% to about 1% by weight of the topical composition.

In some embodiments, the topical composition dries quickly after application. For example, in some embodiments, the topical composition dries within 120 seconds, within 90 seconds, within 80 second, within 70 seconds, within 60 seconds, or within 30 seconds after application.

In some embodiments, the sprayable topical composition consists essentially of (a) about 0.05% to about 5% by weight naftifine or pharmaceutical acceptable salt thereof, (b) a film forming agent comprising octylacrylamide acrylate copolymer, (c) a propellant, (d) an alcoholic solvent, and (e) penetration enhancer. In some embodiments, the topical composition above does not comprise a hydrophilic polymer, e.g., a hydrophilic film-forming polymer.

In some embodiments, the sprayable topical composition comprises (a) about 0.05% to about 5% by weight naftifine or pharmaceutical acceptable salt thereof, (b) about 1% to about 5% by weight of a film forming agent comprising octylacrylamide acrylate copolymer, (c) about 25% to about 45% by weight propellant, (d) about 50% to about 70% by weight alcoholic solvent, and (e) about 1% to about 3.5% by weight penetration enhancer.

The topical composition of the present invention can be administered by means known in the art. In some embodiments, the disclosure provides a delivery device for administering the composition. In some embodiments, the disclosure provides a delivery device comprising the topical compositions as described herein. In some embodiments, the delivery device is a sealed and pressurized device, e.g., an aerosol container. In some embodiments, the delivery device is a sealed and pressurized device, an aerosol container, with a means for providing an aerosol spray of the topical composition, e.g., an aerosol nozzle. In some embodiments, the delivery device comprises an amount of the topical composition sufficient for a single administration of the composition. In some embodiments, the delivery device comprises an amount of the topical composition sufficient for two administrations of the composition. In some embodiments, the delivery device comprises an amount of the topical composition sufficient for three or more administrations of the composition. In some embodiments, the disclosure provides a kit, wherein the kit comprises one or more, e.g., two, delivery devices. In one embodiment, the kit comprises two, three, four, five, six or more delivery devices, wherein one delivery device is used in its entirety for each administration. In some embodiments, the kit comprises two aerosol canister comprising the topical compositions described herein, wherein one aerosol canister is used in its entirety for each administration. In some embodiments, the kit comprises one aerosol canister comprising the topical compositions described herein, in an amount sufficient to fully cover both feet. In some embodiments, the kit comprises two aerosol canisters comprising the topical compositions described herein, each canister is in an amount sufficient to fully cover one feet.

In embodiments in which the delivery device is a pressurized device, the pressure in the sealed and pressurized device can be any pressure suitable for delivery of the topical composition. In some embodiments, the pressure is about 28 psi to about 145 psi at 25° C.

In some embodiments, the delivery device comprising the topical composition is an aerosol device, with a means for providing an aerosol of the topical composition. As used herein, the aerosol device would include any device which is capable of producing fine aerosol droplets of the topical composition. In some embodiments, the aerosol device produces aerosol droplets of the topical composition with an average diameter of about 20 μm to about 150 μm. In some embodiments, the aerosol device produces aerosol droplets of the topical composition with an average diameter of less than 300 μm, less than 250 μm, less than 200 μm, less than 150 μm, less than 100 μm, less than 50 μm, or less than 25 μm. In some embodiments, at least 99% of droplets are 100 μm and above in diameter. In some embodiments, at least 99% of droplets are 100 μm and below in diameter.

In some embodiments, the disclosure provides a product of manufacture for the treatment of a dermatophyte skin infection, e.g., Tinea pedis or onychomycosis in a subject in need thereof, comprising (a) a sealed, pressurized container comprising (i) the topical composition as described herein, and (ii) means for providing an aerosol spray of the topical composition, and (b) instructions for use of the topical composition for treating the dermatophyte skin infection.

### Drug-Device Combination

In some embodiments, the disclosure provides a drug-device combination for dispensing a composition, the drug-device combination comprising a container comprising a composition comprising naftifine or pharmaceutical acceptable salt thereof, a film forming agent comprising octylacrylamide acrylate copolymer, an alcoholic solvent, and a propellant.

In some embodiments, the disclosure provides a drug-device combination for dispensing a composition, the drug-device combination comprising a container comprising a composition comprising about 0.05% to about 5% by weight naftifine or pharmaceutical acceptable salt thereof, a film forming agent comprising octylacrylamide acrylate copolymer, an alcoholic solvent, and a propellant, wherein the ratio of the alcoholic solvent to propellant in the composition is about 1:1 to about 3:1 (w/w).

In some embodiments, the disclosure provides a drug-device combination for dispensing a composition, the drug-device combination comprising a container comprising a composition comprising naftifine or pharmaceutical acceptable salt thereof, a film forming agent comprising octylacrylamide acrylate copolymer, an alcoholic solvent, and a propellant, wherein the ratio of the alcoholic solvent to propellant in the composition is about 1:1 to about 3:1 (w/w).

In some embodiments, the disclosure provides a drug-device combination for dispensing a composition, the drug-device combination comprising a container comprising a composition comprising about 0.05% to about 5% by weight naftifine or pharmaceutical acceptable salt thereof, a film forming agent comprising octylacrylamide acrylate copolymer.

In some embodiments, the drug-device combination provides dispensing of the composition from the container. In some embodiments, the drug-device combination provides spraying of the composition from the container. In some embodiments, the drug-device combination provides both spraying and dispensing of the composition from the container. In some embodiments, the drug-device combination forms a pressurized system. In some embodiments, the drug-device combination is a metered dose system. In some embodiments, the drug-device combination is self-administered drug-device combination.

Various containers can be used to hold, store or house the compositions disclosed herein in the drug-device combination. In some embodiments, a container can comprise a metal body, e.g., a body lined with a chemically inert coating material to avoid degradation of the composition due to any interaction between the body and the composition. In some embodiments, a container can comprise a plastic body, e.g., a body lined with a chemically inert coating material to avoid degradation of the composition due to any interaction between the body and the composition. In some embodiments, the container is a substantially rigid metal or plastic container adapted to contain a pressurized propellant located within the container and in contact with the product to be dispensed. In some embodiments, the container is an inner substantially rigid metal or plastic container adapted to contain a pressurized propellant located within an outer container made from the same material and away from contact with the product to be dispensed.

In some embodiments, the container body can be constructed from materials such as metal, glass, ceramics, polyester, polyethylene terephthalate (PET) or other polymers. In some embodiments, glass containers can be provided with a safety coating of, for instance, polypropylene to contain glass shards that may be formed on impact with a hard surface. In some embodiments, metal container bodies can be used to withstand impact and are amenable to surface coating. In some embodiments, the container comprises stainless steel, tinplate and aluminum, or combinations thereof. In some embodiments the aluminum is aluminum alloy or anodized aluminum.

Various inert coating materials can be used to line the container body including any suitable coating material known in the art such as a polymer, lacquer, resin or other coating treatment that creates a barrier between the container and the composition for preventing any chemical interaction between the composition and the container. In some embodiments, the inert material is a non-metallic coating. In some embodiments, known coatings for metal containers include acrylic, phenolic, polyester, epoxy and vinyl resins can be used to line the container body of the drug-device combination disclosed herein. Accordingly, the container coating for use with a composition of the present invention can be selected so that it exhibits no acidic or alkaline reactivity in itself, and that no acidic or alkaline reacting impurities are leached from it in the presence of the composition.

In some embodiments, the interior of a metal container can be lined with materials such as polyamides, polyimides, polypropylene, polyethylene, fluoropolymers, including perfluoroethylenepropylene copolymer (FEP), fluororubber (FPM), ethylene-propylene diene monomer rubber (EPDM), polytetrafluoroethylene (PTFE), ethylene tetrafluoroethylene copolymer (EFTE), perfluoroalkoxyalkanes, perfluoroalkoxyalkylenes, or blends of fluoropolymers with non-fluorocarbon polymers. Fluoropolymers can, for example, be used in combination with polyimide-polyamide resins.

The coating material of the container can be applied as a single layer, or in multiple layers, for example allowing each layer to cure before application of a further layer. The application of more than one coating can be used to shield the composition from the metal container and prevent adhesion of the active ingredients on the container walls.

In some embodiments, the drug-device combination comprises a pressurized system within which is stored a composition to be dispensed as an aerosol. The system can be pressurized, for example, by a propellant which is generally dissolved within the composition. The composition can be released from the container upon opening of, for example, a pressure actuator valve mechanism located in the top of the container.

In some embodiments, the drug-device combinations disclosed herein comprise a valve assembly comprising a valve cup, sometimes referred to as a mounting cup, a valve body or housing provided with a valve stem, a spring, a dip tube and an actuator. In some embodiments, the valve assembly can also include a secondary shut-off valve for controlling flow from the container, whereby composition flow cannot occur through the secondary valve and out of the sprayer when the sprayer is not in use, and contamination or evaporation of the composition in the container accordingly will not occur. In some embodiments, the valve assembly can be constructed such that the operating parts are held together by means of a snap-fit construction. In some embodiments, no screws or bolts and no fastening tools are necessary for construction of the valve assembly. In some embodiments, the parts of the valve assembly are snapped together, piece by piece. In some embodiments, the actuator, also referred to as a trigger sprayer, can include two or more parts. In some embodiments, the actuator can include a trigger integrally molded with a manifold. In some embodiments, the trigger can also serve as a cap for a body of the actuator. In some embodiments, the trigger and cap can include an integrally formed spring or an integrally formed living hinge. In some embodiments, the spring or living hinge can provide a resistant force when the trigger is actuated and a force sufficient to disengage a manifold from actuation of a valve when forces applied to the trigger during actuation are removed or reduced. In some embodiments, a spring or a living hinge may include geometries which may improve the function of the spring or living hinge. The actuator of the valve assembly can include a body or housing which is affixed to the top of the container body and an actuator plunger or button is fitted within the housing. In some embodiments, the plunger or button can fit over, or within the body in some manner, and connects with or at least indirectly engages or actuates the valve stem.

In some embodiments, an actuator can be actuated by a squeezable actuator. In some embodiments, a pull tab can be used to actuate an actuator. In some embodiments, an actuator may include an extendable handle. In other embodiments, an actuator may include a timer. In other embodiments, an actuator can include an integrated hood or wind shield. In some embodiments, an actuator can also include selectable spray patterns. In some embodiments, an actuator can also include a locking mechanism.

In some embodiments, actuators can be made of any desired material or combination of materials. In some embodiments, an actuator may be made of plastic or a resin-based material. In some embodiments, an actuator can include metal components. In other embodiments, flexible, expandable, or other elastomeric-type materials may be used or integrated into an actuator design. In some embodiments, actuators may be attached to, mated with, or otherwise assembled with an a plastic or metal container having a valve.

In some embodiments, actuators are coupled to a protruding stem of a valve mounted in the container and are adapted to be depressed by the finger of a user in order to operate the associated valve and thereby allow release of composition from the container. Optional materials for the valve stem are polyamide and acetal copolymers.

In some embodiments, the valve stem can be disposed such that the valve stem emerges through a central opening in a pedestal portion of a mounting cup. In some embodiments, beneath the underside of the pedestal is a gasket which encircles a lateral orifice in the hollow valve stem and acts to seal the orifice in the valve stem when the valve is in a closed position. The stem gasket can be selected to survive extreme chemical conditions and not soften, harden or crack within the valve. Any of these changes in the material may cause improper valve functioning and/or loss of seal and product leakage. In some embodiments, the stem gasket is formed from rubber or other elastomeric material and is dimensioned to seal against the outer surface of the valve stem.

A product passage is generally provided for guiding the released, dispensed or sprayed composition out of the container and through the valve actuation assembly to the desired environment. In some embodiments, the drug-device combinations disclosed herein comprise a product passage. In some embodiments, the product passage of the drug-device combination disclosed herein is a dip tube. In some embodiments, the dip tube can be integral with either the container body or a button, or be a separate structure which communicates between the valve stem and a product dispensing orifice formed in the actuator. In some embodiments, the dip tube extends through a neck of the container and into fluid contents of the container. In some embodiments, the dip tube fluidly communicates the container with the fluid supply passageway of the valve assembly. In some embodiments, the dip tube is a separate straw-like element. In some embodiments, the dip tube is produced from low-density polyethylene, medium-density polyethylene, or high-density polyethylene or polypropylene thermoplastic materials.

The liquid composition to be dispensed or sprayed from the container can be caused to fill a pressure chamber and, as it is filled, push against a pressure piston that is supported by a spring that is provided in the pressure chamber. Thus, when a user pumps liquid into the pressure chamber this liquid pushes on the pressure piston, which loads (compresses) the spring, and thus puts the liquid in the pressure chamber under pressure in a manner similar to the pressurized contents of container. In exemplary embodiments, such a pressure spring can be a spring in the broadest sense, and thus can be any resilient device which can store potential energy, including, for example, an air or gas shock absorber or spring, a spring of various compositions and materials, and the like.

Spray characterization, e.g., droplet size distribution (DSD) of the plume released subsequent to spray, can be measured under specified experimental and instrumental conditions by appropriate analytical procedures and validated and/or calibrated known in the art. This includes, but is not limited to, photography, laser diffraction and impaction systems (waterfall impaction, next generation impaction (NGI), etc. The droplet size distribution can be controlled in terms of intervals for D₉₀ percentage of droplets greater than 10 mm. Particle diameter designations “(D)” refer to the representative diameter where 90% (D₉₀), 50% (D₅₀), and 10% (D₁₀) of the total volume of spray liquid is composed of droplets with diameters smaller than or equal to the declared value. Spray characteristics can refer to an average or median value collected for a plurality of sprays. The plurality of sprays can be at least 2, 3, 4, 5, 6, 7, 8, 9 or 10 or more sprays.

In some embodiments, the drug-device combination is suitable for providing a spray comprising the composition, wherein the spray has a D₉₀ droplet size greater than 1 μm, greater than 10 μm, greater than 20 μm, greater than 30 μm, greater than 50 μm, greater than 70 μm, or greater than 100 μm. In some embodiments, the drug-device combination is suitable for providing a spray comprising the composition, wherein the spray has a D₉₀ droplet size less than 500 μm, less than 400 μm, less than 300 μm, or less than 200 μm. In some embodiments, the composition has a D₉₀ droplet size of about 140 μm to about 170 μm when sprayed from drug-device combination. In some embodiments, the composition has a D₉₀ droplet size of about 140 μm to about 145 μm when sprayed from drug-device combination. In some embodiments, the composition has a D₉₀ droplet size of about 145 μm to about 150 μm when sprayed from drug-device combination. In some embodiments, the composition has a D₉₀ droplet size of about 140 μm to about 150 μm when sprayed from drug-device combination. In some embodiments, the composition has a D₉₀ droplet size of about 140 μm to about 160 μm when sprayed from drug-device combination. In some embodiments, the composition has a D₉₀ droplet size of about 150 μm to about 160 μm when sprayed from drug-device combination. In some embodiments, the composition has a D₉₀ droplet size of about 150 μm to about 165 μm when sprayed from drug-device combination. In some embodiments, the composition has a D₉₀ droplet size of about 160 μm to about 165 μm when sprayed from drug-device combination. In some embodiments, the composition has a D₉₀ droplet size of about 165 μm to about 170 μm when sprayed from drug-device combination.

In some embodiments, the composition has an average D₉₀ droplet size of about 155 μm to about 163 μm when sprayed from drug-device combination. In some embodiments, the composition has an average D₉₀ droplet size of about 155 μm to about 160 μm when sprayed from drug-device combination. In some embodiments, the composition has an average D₉₀ droplet size of about 160 μm to about 163 μm when sprayed from drug-device combination. In some embodiments, the composition has an average D₉₀ droplet size of about 155 μm to about 157 μm when sprayed from drug-device combination. In some embodiments, the composition has an average D₉₀ droplet size of about 157 μm to about 160 μm when sprayed from drug-device combination.

In some embodiments, the naftifine or pharmaceutical acceptable salt thereof in the composition of the drug-device combination is naftifine hydrochloride. The concentration of naftifine can vary in the composition of the drug-device combination. In some embodiments, the concentration of naftifine in the composition of drug-device combination is less than 5% by weight of the composition. In some embodiments, the concentration of naftifine in the composition of drug-device combination is less than 4.5%, less than 4.0%, less than 3.5%, or less than 3.0% by weight of the composition. In some embodiments, the composition of the drug-device combination comprises about 0.05% to about 5% by weight naftifine. In some embodiments, the composition of the drug-device combination comprises about 0.05% to about 4%, about 0.1% to about 3.5%, about 0.1% to about 3.0%, about 0.2% to about 3.0%, about 0.5% to about 2.5%, about 1.0% to about 3.0%, about 1.0% to about 2.5%, about 1.5% to about 2.5%, about 1.8% to about 2.2% or about 2.0% by weight naftifine.

The present disclosure provides drug-device combinations comprising a topical composition for administration. In some embodiments, the present disclosure provides an aerosol spray composition for administration from a drug-device combination. In some embodiments, the term aerosol spray composition is a pressurized composition, e.g., a composition in a closed container under pressure, such that an aerosol can be formed upon release form the container in the drug-device combination. In some embodiments, a drug-device combination can include a composition with or without being pressurized. For example, the term “topical composition” can include (i) a liquid composition inside a pressurized container, which becomes an aerosol spray when discharged from the container, and (2) the aerosol spray discharged from the container. Additionally, the term “topical composition” in some embodiments can include the liquid composition on a surface, e.g., a skin surface, which forms when the aerosol spray comes in contact with the surface. In some embodiments, administration of the aerosol spray from the drug-device combination provides a more consistent administration of the composition on the subject being treated. In some embodiments, administration of the aerosol spray from the drug-device combination provides a more thorough administration of the composition to the subject, e.g., more area is covered. In some embodiments, administration of the aerosol spray from the drug-device combination provides a more convenient method of administration. In some embodiments, administration of the aerosol spray from the drug-device reduces the need for other applicators, e.g., wipes, gauzes, clothes, etc. In some embodiments, administration of the aerosol spray from the drug-device combination reduces exposure of the hands or other unintended body parts to the presence of naftifine that may occur by administration by creams, gels, or liquids. In some embodiments, administration of the aerosol spray from the drug-device combination provides a more convenient mode of administration, as it allows the composition to dry quickly after application.

### Methods of Treatment

The topical compositions and drug-device combinations described herein can be used in the treatment of a dermatophyte skin infection. The term “dermatophyte skin infection” can refer to any skin condition caused by dermatophyte fungi, including, e.g., Microsporum, Trichophyton, and Epider-mophyton fungal species. In some embodiments, the term “dermatophyte skin infection” refers to Tinea pedis, interdigital Tinea pedis, Tinea cruris, Tinea corporis, and/or Tinea capitis. In some embodiments, the term “dermatophyte skin infection” refers to Tinea pedis, interdigital Tinea pedis or Tinea cruris. In some embodiments, the disclosure provides a method of treating a dermatophyte skin infection in a subject in need thereof, the method comprising topically administering to the subject the topical composition of any topical spray composition described herein.

The topical compositions and drug-device combinations described herein can be used in the treatment of onychomycosis. The term “onychomycosis” refers to a fungal infection of one or more nails, e.g., toe nails on a subject, resulting in discoloration, thickening, and/or separation of the nail from the nail bed. In some embodiments, onychomycosis refers to brittle, crumbly or ragged nails caused by a fungal infect. In some embodiments, onychomycosis refers to a distorted shape of a nail caused by a fungal infection. In some embodiments, the onychomycosis started from a fungal infection in the skin, e.g., from Tinea pedis. In some embodiments, the onychomycosis is caused by a weakened immune system, reduced blood circulation (e.g., as a result of diabetes), or old age.

The term “treatment” and “alleviation” of the dermatophyte skin infection as used herein refers to reducing the severity of a symptom associated with a dermatophyte skin infection. Thus, in some embodiments, methods provided herein provide palliative care. In some embodiments, the term “treatment” refers to eliminating a symptom associated with a dermatophyte skin infection. Thus, in some embodiments, the methods provided herein provide curative care. In some embodiments, the symptoms associated with a dermatophyte skin infection include one or more of itching, burning, whitening or breakdown of the skin, scaling, blistering, inflammation, pustules, or ulcers. In some embodiments, the methods described herein reduce and/or eliminate the severity one, two, three or more than three symptoms associated with a dermatophyte skin infection.

The term “treatment” and “alleviation” of the onychomycosis as used herein refers to reducing the severity of a symptom associated with onychomycosis. Thus, in some embodiments, methods provided herein provide palliative care for onychomycosis. In some embodiments, the term “treatment” refers to eliminating a symptom associated with onychomycosis. Thus, in some embodiments, the methods provided herein provide curative care associated with onychomycosis. In some embodiments, the methods described herein reduce and/or eliminate the severity one, two, three or more than three symptoms associated with onychomycosis.

In some embodiments, the methods of treatment described herein provide an increased mycological cure rate. The term mycological cure rate as used herein indicates the percentage of collected specimens (skin scrapings from the Tinea pedis lesion) having (a) negative KOH stain following microscopic examination; and (b) negative fungal culture results.

In some embodiments, the composition has a mycological cure rate of at least 20% with a single-dose treatment, at least 30% with a single-dose treatment, at least 40% with a single-dose treatment, at least 50% with a single-dose treatment, at least 60% with a single-dose treatment, at least 70% with a single-dose treatment, or at least 80% with a single-dose treatment. In some embodiments, the composition has a mycological cure rate of at least 20% with a two-dose treatment, at least 30% with a two-dose treatment, at least 40% with a two-dose treatment, at least 50% with a two-dose treatment, at least 60% with a two-dose treatment, at least 70% with a two-dose treatment, or at least 80% with a two-dose treatment. The mycological cure rate is determined by defining cure as negative KOH stain using microscopy and negative fungal culture of skin scrapings taken from the lesion six weeks after the treatment.

In some embodiments, the composition has a Complete Clinical Cure rate of at least 20% with a single-dose treatment, at least 30% with a single-dose treatment, at least 40% with a single-dose treatment, at least 50% with a single-dose treatment, at least 60% with a single-dose treatment, at least 70% with a single-dose treatment, or at least 80% with a single-dose treatment. In some embodiments, the composition has a Complete Clinical Cure rate of at least 20% with a two-dose treatment, at least 30% with a two-dose treatment, at least 40% with a two-dose treatment, at least 50% with a two-dose treatment, at least 60% with a two-dose treatment, at least 70% with a two-dose treatment, or at least 80% with a two-dose treatment. The Complete Clinical Cure rate is defined as an absence in erythema, scaling and pruritus six weeks after the treatment.

In some embodiments, the composition has a Therapeutic Effectiveness rate of at least 20% with a single-dose treatment, at least 30% with a single-dose treatment, at least 40% with a single-dose treatment, at least 50% with a single-dose treatment, at least 60% with a single-dose treatment, at least 70% with a single-dose treatment, or at least 80% with a single-dose treatment. In some embodiments, the composition has a Therapeutic Effectiveness rate of at least 20% with a two-dose treatment, at least 30% with a two-dose treatment, at least 40% with a two-dose treatment, at least 50% with a two-dose treatment, at least 60% with a two-dose treatment, at least 70% with a two-dose treatment, or at least 80% with a two-dose treatment. The Therapeutic Effectiveness is defined as negative KOH stain, negative fungal culture and absent or mild erythema, scaling and pruritus six weeks after the treatment.

In some embodiments, the composition has a Complete Clearance rate of at least 20% with a single-dose treatment, at least 30% with a single-dose treatment, at least 40% with a single-dose treatment, at least 50% with a single-dose treatment, at least 60% with a single-dose treatment, at least 70% with a single-dose treatment, or at least 80% with a single-dose treatment. In some embodiments, the composition has a Complete Clearance rate of at least 20% with a two-dose treatment, at least 30% with a two-dose treatment, at least 40% with a two-dose treatment, at least 50% with a two-dose treatment, at least 60% with a two-dose treatment, at least 70% with a two-dose treatment, or at least 80% with a two-dose treatment. The Complete Clearance is defined as negative KOH stain, negative fungal culture and complete absence of erythema, scaling and pruritus six weeks after the treatment One of skill in the art will appreciate that not all subjects will respond equally to the methods described herein. Thus, when referring to a treatment reducing and/or eliminating a symptom, the skilled artisan can appreciate any statistical significance of clinical results related to the present invention, and are within the scope of the invention. The skilled artisan will also appreciate that “treatment” may be dependent on the frequency of administration, the amount of active agent administered, the disposition of the subject being treated (e.g., age, condition, sex, height, weight, underlying health conditions, etc.). In some embodiments, the methods provided herein provide “treatments” for a dermatophyte skin infection or onychomycosis in a population of subjects, wherein the treatment provides for a reduction in the severity of a symptom associated with a dermatophyte skin infection or onychomycosis, and/or the elimination of a symptom associated with a dermatophyte skin infection or onychomycosis, for greater than 30%, greater than 40%, greater than 50%, greater than 60%, or greater than 70% of the subjects in the population.

In some embodiments, the topical composition is administered to the site of the infection, as well as to the healthy surrounding skin. In some embodiments, the topical composition is dispensed from an aerosol canister as a topical film-forming spray applied to both feet on a single occasion (per single application), even if only one foot has signs and symptoms of infection. The single continuous spray is intended to cover the lesioned, and healthy adjacent skin between and around the toes as well as the bottom and sides of each foot. For example, in some embodiments, the topical composition is applied such that the entire affected area is covered by the topical composition. In some embodiments, greater than 30%, greater than 40%, greater than 50%, greater than 60%, greater than 70%, greater than 80%, greater than 90% or greater than 95% of the affected area is covered by the topical composition. In some embodiments, when the infection affects a specific body part, e.g., a foot, then the topical composition is applied to the entirety of the body part. For example, in some embodiments, the topical composition can be applied to an area exceeding the affected area. For example, in some embodiments, when the dermatophyte skin infection affects the foot, then the topical composition can be applied to greater than 30%, greater than 40%, greater than 50%, greater than 60%, greater than 70%, greater than 80%, greater than 90% or greater than 95% of the foot. The same would apply to any other specific body part affected by a dermatophyte skin infection. Therefore, in some embodiments, the topical composition can cover two feet even without infection on the applied area.

In some embodiments, the present disclosure provides for topical compositions that require a reduced frequency of administrations for treatment of a dermatophyte skin infection. For example, in some embodiments, the composition is administered not more than once in a three day period. In some embodiments, the composition is administered not more than once in a four day period, not more than once in a five day period, not more than once in a six day period, not more than once in a seven day period (1 week), not more than once in a ten day period, or not more than once in a fourteen day period (2 weeks). In some embodiments, the composition is administered not more than once.

In some embodiments, the present disclosure provides for topical compositions that require a reduced number of administrations for treatment of a dermatophyte skin infection or onychomycosis. In some embodiments, the topical composition is administered not more than one time in the course of treatment to the subject to alleviate the dermatophyte skin condition or onychomycosis. In some embodiments, the topical composition is administered not more than two times to the subject to alleviate the dermatophyte skin condition or onychomycosis. In some embodiments, the topical composition is administered not more than three times, four times, or five times to the subject to alleviate the dermatophyte skin condition or onychomycosis.

In some embodiments, wherein more than one administration is required, various administration regimens can be used. In some embodiments, the methods described herein comprise a first administration of the topical composition and a second administration of the topical composition. In some embodiments, the second composition is 5 days to 10 days after the first administration. In some embodiments, the second composition is 6 to 9 days, 7 to 8 days, or about 7 days after the first administration.

The effectiveness of treatment of the dermatophyte skin condition can be determined by methods known to the skilled artisan. For example, in some embodiments, the effectiveness of treatment of the dermatophyte skin condition can be determined by (a) comparing the clinical signs and symptoms associated with Tinea pedis in (i) a subject treated with the topical composition to; (ii) a subject treated with a placebo treatment; and/or (b) comparing mycological cure rate (i.e., potassium hydroxide stain and fungal culture results) in (i) a subject treated with the topical composition to; (ii) a subject treated with a placebo.

In some embodiments, efficacy is measured using mycological cure (KOH and fungal culture), therapeutic effectiveness (defined as negative KOH stain, negative fungal culture and absent or mild erythema, scaling and pruritus) and clinical cure (defined as the absence of erythema, scaling and pruritus. In some embodiments, the primary efficacy endpoint is achieving Complete Clearance at Day 42 defined as negative KOH, negative fungal culture, and the absence of the individual signs and symptoms (i.e., erythema, scaling and pruritus). In some embodiments, a percentage of subjects being administered the topical composition achieves a primary efficacy endpoint that is double a percentage of subjects being administer a placebo or not being treated.

In some embodiments, the topical compositions provide for low naftifine systemic exposure. In some embodiments, the systemic levels of naftifine following a single application the topical spray composition are about 10 pg/mL to about 250 pg/mL, or about 50 pg/mL to about 150 pg·mL on average after 4 weeks. In some embodiments, the systemic levels of naftifine following a single application the topical spray composition are about 100 pg/mL on average after 4 weeks.

In some embodiments, the disclosure provides a method of treating a dermatophyte skin infection in a patient, the method comprising administering two doses of a topical composition comprising naftifine to the subject, wherein the topical composition is a monophasic solution and the naftifine is present in at least 80% saturation under conditions of use, wherein the second dose is administered about 1 week after first dose. In some embodiments, the dermatophyte skin condition is Tinea pedis, interdigital Tinea pedis or Tinea cruris. In some embodiments, the disclosure provides a method of treating onychomycosis in a patient, the method comprising administering two doses of a topical composition comprising naftifine to the subject, wherein the topical composition is a monophasic solution and the naftifine is present in at least 80% saturation under conditions of use, wherein the second dose is administered about 1 week after first dose.

In some embodiments, the disclosure provides a method for maintaining a therapeutic level of naftifine in the stratum corneum in a subject to alleviate the symptoms associated with a dermatophyte skin condition. In some embodiments, the therapeutic level of naftifine in the stratum corneum is measured using a stripping assay. In some embodiments, the therapeutic level of naftifine comprises having a naftifine tape stripping concentration of greater than 10,000 pg/mL, greater than 12,000 pg/mL, or greater than 15,000 pg/mL. In some embodiments, the therapeutic level is at least 10,000 pg/mL. In some embodiments, the therapeutic level is maintained for at least 1 week, at least 2 weeks, at least 3 weeks or at least 4 weeks.

In some embodiments, the disclosure provides a method for maintaining a therapeutic level of naftifine in the stratum corneum in a subject for four weeks, the method comprising administering two doses of a topical composition comprising naftifine to the subject, wherein the composition is a monophasic solution and the pharmaceutical is present in at least 80% saturation under conditions of use, wherein the second dose is administered about 1 week after first dose.

In some embodiments, the amount of naftifine in the stratum corneum of the subject four weeks after the first dose and three weeks following the second dose is on average half the amount extracted at the second dose.

In some embodiments, the disclosure provides a method of treating a dermatophyte skin infection in a subject in need thereof, the method comprising administering a topical composition, wherein the administration of the topical composition to the subject results in at least 10% success, at least 20% success, at least 30% success, at least 40% success, at least 50% success, at least 60% success, at least 70% success or at least 80% success in an efficacy endpoint.

Various efficacy endpoints are known to the skilled artisan, of which the main efficacy endpoint in clinical trials is achieving Complete Clearance. In some embodiments, Complete Clearance is measured at day 21, day 28, day 35, day 42, day 47 or day 54. In some embodiments, Complete clearance is measured at Day 42. Complete clearance can be defined as negative KOH, negative fungal culture, and/or scores of 0 ‘none’ for the individual signs and symptoms selected from erythema, scaling and pruritus. In some embodiments, the efficacy endpoint is achieving Complete Clearance at Day 42 defined as negative KOH, negative fungal culture, and/or scores of 0 ‘none’ for the individual signs and symptoms selected from erythema, scaling and pruritus.

In some embodiments, the disclosure provides a method of treating a dermatophyte skin infection in a subject in need thereof, the method comprising administering a topical composition comprising 2% naftifine or its pharmaceutically acceptable salt, wherein the topical composition is administered to the subject in two doses, wherein the second dose is administered about 1 week after the first dose, wherein the administration of the topical composition to the subject results in at least 50% success in an efficacy endpoint. In some embodiments, the dermatophyte skin condition is Tinea pedis, interdigital Tinea pedis or Tinea cruris.

In some embodiments, the topical compositions are administered to a population of subjects. In some embodiments, a percentage of subjects being administered the topical composition achieves a primary efficacy endpoint that is double a percentage of subjects being administered a placebo.

In some embodiments, the primary efficacy endpoint is a measure of mycological cure, therapeutic effectiveness and complete clearance defined as negative KOH, negative fungal culture, and the absence of individual signs and symptoms selected from erythema, scaling and pruritus.

In some embodiments, the disclosure provides a method of treating onychomycosis in a subject in need thereof, the method comprising administering a topical composition, wherein the administration of the topical composition to the subject results in at least 10% success, at least 20% success, at least 30% success, at least 40% success, at least 50% success, at least 60% success, at least 70% success or at least 80% success in an efficacy endpoint. Onychomycosis is a fungal infection of the nails that causes discoloration, thickening, and separation from the nail bed.

In some embodiments, the efficacy endpoint associated with onychomycosis is the at least 50% reduction, at least 80% reduction or at least 90% reduction of one or more symptoms associated with onychomycosis, e.g. discoloration, thickening, and/or separation of the nail from the nail bed, brittleness, crumbliness or ragged nails. In some embodiments, the efficacy endpoint is determined 1 week, 2 weeks, 3 weeks, 4 weeks, 5 weeks or 6 weeks after the terminal administration of the topical spray.

In some embodiments, the efficacy endpoint associated with mycological cure is greater than 40%, greater than 50%, or greater than 60%. In some embodiments, the efficacy endpoint associated with mycological cure is about 40% to about 80% or about 50% to about 70%.

In some embodiments, the efficacy endpoint associated with complete clinical cure is greater than 20%, greater than 25%, or greater than 300%. In some embodiments, the efficacy endpoint associated with mycological cure is about 20% to about 50% or about 25% to about 40%.

In some embodiments, the efficacy endpoint associated with therapeutic effectiveness is greater than 40%, greater than 45%, or greater than 50%. In some embodiments, the efficacy endpoint associated with therapeutic effectiveness is about 40% to about 80% or about 45% to about 70%.

In some embodiments, the efficacy endpoint associated with complete clearance is greater than 5%, greater than 10%, or greater than 50%. In some embodiments, the efficacy endpoint associated with mycological cure is about 5% to about 40% or about 10% to about 35%.

In some embodiments, the disclosure provides a method of treating a dermatophyte skin infection in a subject, the method comprising administering a topical composition comprising naftifine, wherein the composition is a monophasic solution and the pharmaceutical is present in at least 80% saturation under conditions of use, wherein the administration of the topical composition to the subject results in a complete clearance of signs and symptoms of Tinea pedis, wherein complete clearance is defined as achieving the following scores for all clinical sign and symptom listed below:
(a) a decrease in severity of erythema from baseline with a score of 0 on day 42;
(b) a decrease in severity of scaling from baseline with a score of 0 on day 42;
(c) a decrease in severity of pruritus from baseline with a score of 0 on day 42;
   and achieving mycological clear in KOH test and in mycological culture.

In some embodiments, the dermatophyte skin condition is Tinea pedis, interdigital tinea pedis or Tinea cruris.

In some embodiments, the Arithmetic Mean AUCₜ(pg*h/mL) of Naftifine following a single application of the Naftifine topical composition is not more than 300,000 pg*h/mL, not more than 250,000 pg*h/mL, or not more than 200,000 pg*h/mL,

In some embodiments, the Cₘₐₓ(pg/mL) of Naftifine following a single application of the Naftifine topical composition is not more than 3000 pg/mL, not more than 2500 pg/mL, not more than 2000 pg/mL, not more than 1500 pg/mL, or not more than 1000 pg/mL,

In some embodiments, the Median Tmax (h) of Naftifine following a single application of the Naftifine topical composition is 12h, 18h, 24 h, 30h or 36h.

In some embodiments, the present disclosure provides a method of treating a dermatophyte skin infection or onychomycosis in a subject in need thereof, the method comprising applying an aerosol spray or a mist to the skin using the drug-device combinations disclosed herein. In some embodiments, the dermatophyte skin condition is Tinea pedis or interdigital Tinea pedis or Tinea cruris.

In some embodiments, the composition of the drug-device combination is administered to the site of the infection. For example, in some embodiments, the composition of the drug-device combination is applied such that the entire affected area is covered by the composition using the spray droplet size (D₉₀), as described herein. In some embodiments, greater than 30%, greater than 40%, greater than 50%, greater than 60%, greater than 70%, greater than 80%, greater than 90% or greater than 95% of the affected area is covered by the composition using the spray droplet size (D₉₀), as described herein.

In some embodiments, the present disclosure provides for compositions administered from the drug-device combinations described herein that require a reduced frequency of administrations for treatment of a dermatophyte skin infection. For example, in some embodiments, the composition is administered not more than once in a three day period using the spray droplet size (D₉₀), as described herein. In some embodiments, the composition is administered not more than once in a four day period, not more than once in a five day period, not more than once in a six day period, not more than once in a seven day period (1 week), not more than once in a ten day period, or not more than once in a fourteen day period (2 weeks) using the spray droplet size (D₉₀), as described herein.

In some embodiments, the present disclosure provides for compositions dispensed from a drug-device combination using the spray droplet size (D₉₀), as described herein, that require a reduced number of administrations for treatment of a dermatophyte skin infection or onychomycosis. In some embodiments, the composition is administered using the spray droplet size (D₉₀), as described herein, such that not more than one time in the course of treatment to the subject is sufficient to alleviate the dermatophyte skin condition or onychomycosis. In some embodiments, the composition is administered not more than two times to the subject to alleviate the dermatophyte skin condition or onychomycosis. In some embodiments, the composition is administered not more than three times, four times, or five times to the subject to alleviate the dermatophyte skin condition or onychomycosis.

In some embodiments, wherein more than one administration is required, various administration regimens can be used. In some embodiments, the methods described herein comprise a first administration of the composition and a second administration of the composition. In some embodiments, the second composition is 5 days to 10 days after the first administration. In some embodiments, the second composition is 6 to 9 days, 7 to 8 days, or about 7 days after the first administration.

It is important for the composition to quickly dry after application to prevent dripping and runoff. In some embodiments, the composition of the drug-device combination dries quickly after application. In some embodiments, the composition dries less than three minutes after application to the skin. In some embodiments, the composition dries less than two minutes after application to the skin. In some embodiments, the composition dries in less than one minute after application to the skin. For example, in some embodiments, the composition of the drug-device combination using the spray droplet size (D₉₀), as described herein. dries within 120 seconds, within 90 seconds, within 80 second, within 70 seconds, within 60 seconds, or within 30 seconds after application. In some embodiments, the composition dries within 90 seconds, within 80 seconds, within 70 seconds or within 60 seconds after application of 500 μL, 250 μL, 100 μL or 50 μL of the composition to the skin at room temperature and 1 atmospheric pressure.

All references cited herein, including patents, patent applications, papers, textbooks and the like, and the references cited therein, to the extent that they are not already, are hereby incorporated herein by reference in their entirety.

### EXAMPLES

### Example 1—Topical Naftifine Compositions:Various Film Forming Agents

Various topical compositions comprising 2% naftifine were made as outlined in Table 1 and Table 2.

TABLE 1

| | Sample # | | | | | | | |
|---|---|---|---|---|---|---|---|---|
| Component | 1 | 2 | 3 | 4 | 5 | 6 | 7 | 8 |
| Propylene | 0.75-1.5% | — | 0.75-1.5% | 0.75-1.5% | 0.75-1.5% | 0.75-1.5% | 0.75-1.5% | 0.75-1.5% |
| glycol | | | | | | | | |
| Transcutol ® | — | 0.5-1% | — | 0.5-1% | — | 0.5-1% | 0.5-1% | 0.5-1% |
| Polymer | Methacrylic | Methacrylic | Methacrylic | Methacrylic | Methacrylic | Methacrylic | Methacrylic | Methacrylic |
| | acid- | acid- | acid- | acid- | acid- | acid- | acid- | acid- |
| | methyl acrylate | methyl acrylate | methyl acrylate | methyl acrylate | methyl acrylate | methyl acrylate | methyl acrylate | methyl acrylate |
| | copolymer | copolymer | copolymer | copolymer | copolymer | copolymer | copolymer | copolymer |
| | (Eudragit ® | [symbol] | [symbol] | (Eudragit ® | (Eudragit ® | (Eudragit ® | (Eudragit ® | (Eudragit ® |
| | [symbol] | | | [symbol] | E100) and | E100) | [symbol] | [symbol] |
| | | | | | PVP (K30) | | | |
| | | | | | [symbol] | | | |
| Ethanol | 45-60% | 45-60% | 45-60% | 45-60% | 45-60% | 45-60% | 45-60% | 45-60% |
| Propellant | 35-50% | 35-50% | 35-50% | 35-50% | 35-50% | 35-50% | 35-50% | 35-50% |

[symbol]

indicates data missing or illegible when filed

TABLE 2

| | Sample # | | | | | | |
|---|---|---|---|---|---|---|---|
| Component | 9 | 10 | 11 | 12 | 13 | 14 | 15 |
| Propylene | 0.75-1.5% | 0.75-1.5% | 0.75-1.5% | 0.75-1.5% | 0.75-1.5% | 0.75-1.5% | 0.75-1.5% |
| glycol | | | | | | | |
| Transcutol ® | 0.5-1% | 0.5-1% | 0.5-1% | 0.5-1% | 0.5-1% | 0.5-1% | 0.5-1% |
| Polymer | octylacrylamide | Methacrylic | Methacrylic | Methacrylic | Methacrylic | Butyl Ester of | Butyl Ester of |
| | acrylate | acid- | acid- | acid- | acid- | PVM/MA | PVM/MA |
| | copolymer | methyl acrylate | methyl acrylate | methyl acrylate | methyl acrylate | Copolymer | Copolymer |
| | (Dermacryl) | copolymer | copolymer | copolymer | copolymer | (Gantrez ® | [symbol] |
| | | (Eudragit ® | (Eudragit ® | (Eudragit ® | (Eudragit ® | ES-425) | |
| | | RL100/ | RL100/ | RL100/ | E100/ | | |
| | | [symbol] | [symbol] | [symbol] | [symbol] | | |
| Ethanol | 50-65% | 50-65% | 50-65% | 50-65% | 50-65% | 50-65% | 50-65% |
| Propellant | 30-35% | 30-35% | 30-35% | 30-35% | 30-35% | 30-35% | 30-35% |

[symbol]

indicates data missing or illegible when filed

The samples were then analyzed for (i) visual precipitation, (ii) penetration of naftifine, (iii) antifungal efficacy, (iv) resistance of the resulting film to water and artificial sweat (pH 4.0 and 6.5) 24 hours post application, (v) drying time. The results are presented in Table 3 and Table 4.

TABLE 3

| | Sample # | | | | | | | |
|---|---|---|---|---|---|---|---|---|
| Test | 1 | 2 | 3 | 4 | 5 | 6 | 7 | 8 |
| Penetration rank | — | — | — | — | — | — | 4 | 5 |
| Antifungal Efficacy | — | — | — | — | — | — | 4 | 5 |
| Resistance of film to | water- x | — | water- x | water- + | water- x | water- x | water- + | water- + |
| water and artificial sweat | 4.0- x | | 4.0- x | 4.0- x | 4.0 - x | 4.0 - x | 4.0- + | 4.0 - + |
| | 6.5- x | | 6.5- x | 6.5- x | 6.5 - x | 6.5 - x | 6.5- + | 6.5 - + |
| Drying time | — | — | — | — | — | — | 2 min | — |

TABLE 4

| | Sample # | | | | | | |
|---|---|---|---|---|---|---|---|
| Test | 9 | 10 | 11 | 12 | 13 | 14 | 15 |
| Penetration rank | 1 | — | 3 | — | — | 5 | — |
| Antifungal Efficacy | 1 | — | 2 | — | — | 3 | — |
| Resistance of film to | water- + | water- + | water- + | water- x | water- + | water- x | — |
| water and artificial sweat | 4.0- + | 4.0- x | 4.0- + | 4.0- x | 4.0 - + | 4.0 - x | |
| | 6.5- + | 6.5- x | 6.5- + | 6.5- x | 6.5 - + | 6.5 - x | |
| Drying time | 1 min | — | 70 sec | — | — | — | 150 sec |

The following terms used above can be defined as follows: penetration rank: 1=greatest. Antifungal Efficacy: 1=greatest kill. (i) Resistance: “+” indicates composition was resistant to condition, “x” indicates the composition was not resistant to condition. Tested in water at pH 7.0, pH 4.0, and pH 6.5.

As revealed from Table 3 and Table 4, Sample 9 exhibited the shortest drying time, while having a good antifungal activity and skin penetration and high resistance to both water and artificial sweat.

### Example 2—Topical Naftifine Compositions:Various Solvent:Propellant Ratios

Various topical compositions comprising Naftifine were made by varying the amount of ethanol and propellant as outline in Table 5. The various compositions were sprayed on artificial skin and the following parameters were tested: Appearance of the spray during discharge, Time to film formation (evaluated by the appearance of a film, Full Drying time.

TABLE 5

| | Ethanol:propellant | Appearance of spray | Time to film | Full drying | |
|---|---|---|---|---|---|
| Formulation | ratio (w/w) | during discharge | formation | time | |
| A | Ratio 1.8:1 | Homogeneous aerosol | 16-20 sec | 50-70 | sec |
| | | spray | | | |
| B | Ratio 3.9:1 | Big drops; | 60-65 sec | ~70-85 | sec |
| | | No aerosol pattern | | | |
| C | Ratio 1:1 | No big drops, resembles | 49-58 sec | ~90 | sec |

more the aerosol pattern.

As revealed from Table 5, favorable Time to film formation (evaluated by the appearance of a film and full drying time were obtained with the composition having about 60% ethanol and about 30% propellant, i.e., an alcoholic solvent:propellant ratio of 1.8:1.

### Example 3—Topical Naftifine Compositions

A topical composition (F5) comprising Naftifine was made as outlined in Table 6 based on Sample 9 from Example 1.

TABLE 6

| Ingredient | % w/w |
|---|---|
| Naftifine HCl | 2.0 |
| Ethanol | 50-65% |
| Propylene Glycol | 0.75-1.5% |
| Diethylene Glycol Monoethyl Ether (Transcutol ® P) | 0.5-1% |
| octylacrylamide acrylate copolymer (Dermacryl) | 2-5% |
| 1,1,1,2-tetrafluoroethane (Norflurane ® 134A Propellant) | 30-35% |

### Example 4—Skin Penetration—Tape Stripping Study in Human Subjects

The presence of naftifine in the stratum corneum (SC) was evaluated following 2 applications of F5 (Sample 9 from Example 1) and F3 (Sample 11 from Example 1) on Day 1 and day 8 to the forearm of 10 healthy adults. Skin was tape stripped for quantification of naftifine levels at time points up to 28 days following the initial application. Naftin® Cream, 2% applied once daily for 14 days resulted in higher median naftifine skin levels compared to F5 applied on Days 1 and 8. By 29 days, naftifine levels in the skin following two applications of F5 were at least similar to levels achieved following 2 weeks of daily application with Naftin®. This study demonstrated sustained naftifine levels in the skin that were measurable up to 29 days following the dosing regimen of F5.

TABLE 7
Summary of Naftifine Amounts in Tape Strips by Study Day

| | Total Naftifine Amount (pg) | | | | | | | | |
|---|---|---|---|---|---|---|---|---|---|
| | Day 8 | | | Day 15 | | | Day 29 | | |
| Parameter | F3 | F5 | Naftin ® | F3 | F5 | Naftin ® | F3 | F5 | Naftin ® |
| Mean | 33,623 | 153,162 | 808,159 | 586,973 | 559,453 | 490,694 | 2,757 | 81,504 | 4,036 |
| Median | 22,128 | 73,318 | 393,720 | 104,368 | 134,626 | 303,429 | 1,548 | 3,210 | 3,185 |

F3 and F5 - Single actuation applied topically on Days 1 and 8
Naftin ®, 2% cream (Sebela International Ltd) - Single topical application on Days 1 through 14

### Example 5—Proof of Concept Clinical Study—Naftifine Film-Forming Aerosol, 2%—F5

A randomized, double-blind, placebo-controlled, parallel-group study using F5 as a topical composition was conducted. F5 (N=42) and a placebo (N=40) were administered one time to subjects experiencing interdigital Tinea pedis. On Day 14, Day 28 and Day 42, subjects were rated on the severity of clinical signs and symptoms of disease while on Day 42 mycological cure was assessed. Results on Day 42 of the experiment are shown in Table 8.

TABLE 8

| | Neg. KOH | Erythema, | Erythema, | |
|---|---|---|---|---|
| | and Neg. | Scaling, | Scaling, Pruritus: | |
| Efficacy endpoint | Fungal | Pruritus: | ‘None’(0) or | Δ (%) − F5 over |
| (day 42) | culture | ‘None’ (0) | ‘Mild’ (1) | placebo |
| Mycological cure | + | | | 30% |
| Complete Clinical Cure | | + | | 18% |
| Therapeutic | + | | + | 34% |
| Effectiveness | | | | |
| Complete Clearance | + | + | | 9% |

The clinical results revealed a clear advantage of F5 over placebo in the efficacy endpoint tested.

### Example 6: Dermal Penetration and Toxicity Study in Hanford Minipigs

F1 (Sample 7 from Example 1), F3 (Sample 11 from Example 1), F4 (Sample 15 from Example 1) and F5 were each topically applied twice on Days 1 and 8 to a population of Hanford minipigs. The study yielded no test article-related effects on any organs or systems. Systemic levels of naftifine were detectable in all treated minipigs after a single application (2 ml/kg) as early as 0.5 hours after application, peaking between 6-16 hours after application. Little to no accumulation was observed following a second application of F5, and there was not marked gender differences in dermal uptake with either application. Based on the lack of adverse evidence from the study, all formulations of naftifine at the concentrations tested are considered to be safe in Hanford minipigs when applied as two dermal administrations, one week apart, at a dose level of 2 ml/kg to 10% body surface area. Averaged data from this study is presented below in Table 9 which revealed Lowest systemic absorption with F5 in the minipig animal model,

TABLE 9

| | | F1 | | F3 | | F4 | | F5 | |
|---|---|---|---|---|---|---|---|---|---|
| | | Male | Female | Male | Female | Male | Female | Male | Female |
| Cmax | Day 1 | 11045 | 9513 | 14330 | 19147 | 7916 | 9241 | 5833 | 5677 |
| (pg/mL) | Day 8 | 15089 | 9881 | 10916 | 18537 | 11154 | 8990 | 8916 | 7228 |
| AUC_{0-24 (h*pg/mL)} | Day 1 | 163070 | 148242 | 212248 | 192424 | 105047 | 123476 | 101335 | 81892 |
| | Day 8 | 203364 | 156125 | 178616 | 229963 | 146279 | 168533 | 140769 | 105368 |

### Example 7: Naftifine Levels in Blood Following a Single Application of F5 in Human Subjects

Naftifine levels in blood following a single application of F5 was determined in 7 human subjects. The values obtained were as following: Arithmetic Mean AUCₜ(h*pg/mL)—183,425; Cₘₐₓ (pg/mL)—1051; Median Tmax (h): 24.

### Example 8: Chemical and Physical Stability of the Topical Composition (F5) Comprising Naftifine

The chemical and physical stability parameters of Naftifine composition were evaluated at 25° C. and 60% relative humidity (referred as “RT conditions”), or 40° C. and 75% relative humidity (referred as “accelerated (ACC) conditions”), for a specific duration of TO and 6 months.

The topical composition (F5) comprising Naftifine exhibited both physical stability and chemical stability with no significant change in Naftifine assay (100% at T=0, 99.5% after 6 months in RT and 98.2% after 6 months in accelerated conditions) and Related compounds levels (total) of less than 0.07% at T=0 and after 6 months in RT and in accelerated conditions. See Table 10.

TABLE 10

| | T0 | 6 months (RT) | 6 months (Acc) |
|---|---|---|---|
| Appearance | Homogeneous and | Homogeneous and | Homogeneous and |
| | free of particulate | free of particulate | free of particulate |
| | matter | matter | matter |
| Assay | 100.50% | 99.50% | 98.20% |
| Related compounds: | nd | nd | nd |
| Cis-naftifine | | | |
| Related | nd | nd | nd |
| compounds (total) | | | |

nd: not detected

The data presented demonstrates that Naftifine formulations described herein are not only more stable and suitable for spray administration, but the same formulas are effective in vivo in the treatment of a dermatophyte skin infection. The compositions allow the active to reside on the skin for extended period of time which provides sufficient time for the treatment of a dermatophyte skin infection with a reduced number of administrations, e.g., only one or only two administrations. The one or two administration regimens enhance patient compliance compared to the current regimen of daily administration for 14 days.

### Example 9: Measurement of Droplet Size Distribution of Compositions Sprayed from the Drug-Device Combination

2% Nafitine compositions were prepared according to the formulations disclosed in Example 1. The prepared naftifine compositions were placed and sealed in a drug-device combination described herein. The droplet size distributions (DSD) produced by the spray and aerosol generated by the drug-device combinations were measured using a SprayVIEW® Measurement Systems apparatus. Five actuations of each composition were sprayed at a distance of 15 cm. The mean values of the DSD of the sprays are shown in Table 11 below.

TABLE 11

| Container No. | | D₁₀ | D₅₀ | D₉₀ |
|---|---|---|---|---|
| | 1 | 43.51 | 79.20 | 155.34 |
| | 2 | 42.39 | 78.85 | 158.88 |
| | 3 | 45.02 | 85.52 | 169.46 |
| | 4 | 35.13 | 75.35 | 166.66 |
| | 5 | 41.27 | 74.43 | 147.46 |
| | 6 | 36.92 | 73.85 | 157.00 |
| Mean | | 40.71 | 77.87 | 159.13 |
| % Relative Standard | | | 5.6 | |
| Deviation (RSD) | | | | |

1. A sprayable topical composition comprising:
a. about 0.05% to about 5% by weight naftifine or pharmaceutical acceptable salt thereof,
b. a film forming agent comprising octylacrylamide acrylate copolymer;
c. an alcoholic solvent; and
d. a propellant,
wherein the ratio of the alcoholic solvent to propellant in the composition is about 1:1 to about 3:1 (w/w).

2. (canceled)

3. The topical composition of claim 1, wherein the composition comprises about 1% to about 3% naftifine or a pharmaceutical acceptable salt thereof.

4. (canceled)

5. The topical composition of claim 1, wherein the composition comprises about 2% to about 10% w/w octylacrylamide acrylate copolymer.

6. (canceled)

7. The topical composition of claim 1, wherein the ratio of film forming agent to alcoholic solvent in the composition is about 1:15 to 1:30 (w/w).

8. (canceled)

9. The topical composition of claim 1, wherein the composition comprises less than 4% hydrophilic polymer.

10. (canceled)

11. The topical composition of claim 1, wherein the alcoholic solvent is a C₁-6 alcohol.

12. (canceled)

13. (canceled)

14. The topical composition of claim 1, wherein the composition comprises about 10% to about 70% by weight alcoholic solvent.

15-18. (canceled)

19. The topical composition of claim 1, wherein the propellant is a hydrocarbon propellant, a fluorocarbon propellant, a hydrofluorocarbon propellant, or combination thereof.

20-22. (canceled)

23. The topical composition of claim 1, wherein the composition comprises about 10% to about 50% by weight propellant.

24. (canceled)

25. The topical composition of claim 1, wherein the ratio of the alcoholic solvent to the propellant is about 1.5:1 to about 2.5:1 (w/w).

26-29. (canceled)

30. The topical composition of claim 1, further comprising a penetration enhancer, co-solvent, emulsifier, solubilizer, or combination thereof.

31-42. (canceled)

43. A method of treating a dermatophyte skin infection or onychomycosis in a subject in need thereof, the method comprising topically administering to the subject the topical composition of claim 1.

44. The method of claim 43, wherein the dermatophyte skin condition is Tinea pedis or interdigital Tinea pedis or Tinea cruris.

45. The method of claim 43, wherein the administering comprises applying the topical composition to the subject in an aerosol spray or a mist.

46-53. (canceled)

54. The method claim 43, wherein effectiveness of treatment of the dermatophyte skin condition is determined by
a. comparing (i) a reduction in clinical score of a subject treated with the topical composition to (ii) a reduction in clinical score of a subject treated with a placebo treatment or given no treatment; and/or
b. comparing a reduction in percentage mycological cure observed from (a) a subject treated with the topical composition to (b) a subject treated with a placebo or given no treatment.

55-62. (canceled)

63. A method of treating a dermatophyte skin infection in a subject, the method comprising administering a topical sprayable composition comprising naftifine, wherein the composition is a monophasic solution and the pharmaceutical is present in at least 80% saturation under conditions of use, wherein the administration of the topical composition to the subject results in a complete clearance of clinical signs and symptoms,
wherein the complete clearance is characterized by achieving at least one of the scores for the clinical signs and symptoms listed in (a)-(c):
(a) a decrease in severity of erythema from baseline with a score of 0 on day 42;
(b) a decrease in severity of scaling from baseline with a score of 0 on day 42;
(c) a decrease in severity of pruritus from baseline with a score of 0 on day 42;
and by achieving mycological cure in a KOH test and in mycological culture.

64-68. (canceled)

69. The method of claim 63, wherein the dermatophyte skin condition is Tinea pedis or interdigital Tinea pedis or Tinea cruris.

70-74. (canceled)

75. A drug-device combination for dispensing a composition, the drug-device combination comprising:
a container comprising a composition comprising:
i. naftifine or pharmaceutical acceptable salt thereof;
ii. a film forming agent comprising octylacrylamide acrylate copolymer;
iii. an alcoholic solvent; and
iv. a propellant,
wherein the drug-device combination is suitable for providing a spray comprising the composition, wherein the spray has a D₉₀ droplet size greater than 1 μm.

76-109. (canceled)

110. A method of treating a dermatophyte skin infection or onychomycosis in a subject in need thereof, the method comprising applying an aerosol spray or a mist to the skin using the device of claim 75.

111. The method of claim 110, wherein the dermatophyte skin condition is Tinea pedis or interdigital Tinea pedis or Tinea cruris.

112-115. (canceled)

116. The method according to claim 110, wherein the composition dries less than three minutes after application of the composition to the skin.

117-121. (canceled)
